SEQUENCE LISTING (6 sequences)

SEQ ID NO: 1 - DNA, 3261 bp, Homo sapiens
gggcggggctgcggttgcggtgcctgcgcccgcggcggcggaggcgcaggcggtggcgagtggatatctccggagcatttggataatgtgacagttggaatgcagtgatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaaggcacagagagaatggaagatcagggtcagagtattattccaatgcttactggagaagtgattcctgtaatggaactgctttcatctatgaaatcacacagtgttcctgaagaaatagatatagctgatacagtactcaatgatgatgatattggtgacagctgtcatgaaggctttcttctcaatgccatcagctcacacttgcaaacctgtggctgttccgttgtagtaggtagcagtgcagagaaagtaaataagatagtcagaacattatgcctttttctgactccagcagagagaaaatgctccaggttatgtgaagcagaatcatcatttaaatatgagtcagggctctttgtacaaggcctgctaaaggattcaactggaagctttgtgctgcctttccggcaagtcatgtatgctccatatcccaccacacacatagatgtggatgtcaatactgtgaagcagatgccaccctgtcatgaacatatttataatcagcgtagatacatgagatccgagctgacagccttctggagagccacttcagaagaagacatggctcaggatacgatcatctacactgacgaaagctttactcctgatttgaatatttttcaagatgtcttacacagagacactctagtgaaagccttcctggatcaggtctttcagctgaaacctggcttatctctcagaagtactttccttgcacagtttctacttgtccttcacagaaaagccttgacactaataaaatatatagaagacgatacgcagaagggaaaaaagccctttaaatctcttcggaacctgaagatagaccttgatttaacagcagagggcgatcttaacataataatggctctggctgagaaaattaaaccaggcctacactcttttatctttggaagacctttctacactagtgtgcaagaacgagatgttctaatgactttttaaatgtgtaacttaataagcctattccatcacaatcatgatcgctggtaaagtagctcagtggtgtggggaaacgttcccctggatcatactccagaattctgctctcagcaattgcagttaagtaagttacactacagttctcacaagagcctgtgaggggatgtcaggtgcatcattacattgggtgtctcttttcctagatttatgcttttgggatacagacctatgtttacaatataataaatattattgctatcttttaaagatataataataggatgtaaacttgaccacaactactgtttttttgaaatacatgattcatggtttacatgtgtcaaggtgaaatctgagttggcttttacagatagttgactttctatcttttggcattctttggtgtgtagaattactgtaatacttctgcaatcaactgaaaactagagcctttaaatgatttcaattccacagaaagaaagtgagcttgaacataggatgagctttagaaagaaaattgatcaagcagatgtttaattggaattgattattagatcctactttgtggatttagtccctgggattcagtctgtagaaatgtctaatagttctctatagtccttgttcctggtgaaccacagttagggtgttttgtttattttattgttcttgctattgttgatattctatgtagttgagctctgtaaaaggaaattgtattttatgttttagtaattgttgccaactttttaaattaattttcattatttttgagccaaattgaaatgtgcacctcctgtgccttttttctccttagaaaatctaattacttggaacaagttcagatttcactggtcagtcattttcatcttgttttcttcttgctaagtcttaccatgtacctgctttggcaatcattgcaactctgagattataaaatgccttagagaatatactaactaataagatctttttttcagaaacagaaaatagttccttgagtacttccttcttgcatttctgcctatgtttttgaagttgttgctgtttgcctgcaataggctataaggaatagcaggagaaattttactgaagtgctgttttcctaggtgctactttggcagagctaagttatcttttgttttcttaatgcgtttggaccattttgctggctataaaataactgattaatataattctaacacaatgttgacattgtagttacacaaacacaaataaatattttatttaaaattctggaagtaatataaaagggaaaatatatttataagaaagggataaaggtaatagagcccttctgccccccacccaccaaatttacacaacaaaatgacatgttcgaatgtgaaaggtcataatagctttcccatcatgaatcagaaagatgtggacagcttgatgttttagacaaccactgaactagatgactgttgtactgtagctcagtcatttaaaaaatatataaatactaccttgtagtgtcccatactgtgttttttacatggtagattcttatttaagtgctaactggttattttctttggctggtttattgtactgttatacagaatgtaagttgtacagtgaaataagttattaaagcatgtgtaaacattgttatatatcttttctcctaaatggagaattttgaataaaatatatttgaaattttaaaaaaaaaaaaaaaaaa

SEQ ID NO: 2 - DNA, 30001 bp, Homo sapiens
caaagaaaagggggaggttttgttaaaaaagagaaatgttacatagtgctctttgagaaaattcattggcactattaaggatctgaggagctggtgagtttcaactggtgagtgatggtggtagataaaattagagctgcagcaggtcattttagcaactattagataaaactggtctcaggtcacaacgggcagttgcagcagctggacttggagagaattacactgtgggagcagtgtcatttgtcctaagtgcttttctaccccctacccccactattttagttgggtataaaaagaatgacccaatttgtatgatcaactttcacaaagcatagaacagtaggaaaagggtctgtttctgcagaaggtgtagacgttgagagccattttgtgtatttattcctccctttcttcctcggtgaatgattaaaacgttctgtgtgatttttagtgatgaaaaagattaaatgctactcactgtagtaagtgccatctcacacttgcagatcaaaaggcacacagtttaaaaaacctttgtttttttacacatctgagtggtgtaaatgctactcatctgtagtaagtggaatctatacacctgcagaccaaaagacgcaaggtttcaaaaatctttgtgttttttacacatcaaacagaatggtacgtttttcaaaagttaaaaaaaaacaactcatccacatattgcaactagcaaaaatgacattccccagtgtgaaaatcatgcttgagagaattcttacatgtaaaggcaaaattgcgatgactttgcaggggaccgtgggattcccgcccgcagtgccggagctgtcccctaccagggtttgcagtggagttttgaatgcacttaacagtgtcttacggtaaaaacaaaatttcatccaccaattatgtgttgagcgcccactgcctaccaagcacaaacaaaaccattcaaaaccacgaaatcgtcttcactttctccagatccagcagcctcccctattaaggttcgcacacgctattgcgccaacgctcctccagagcgggtcttaagataaaagaacaggacaagttgccccgccccatttcgctagcctcgtgagaaaacgtcatcgcacatagaaaacagacagacgtaacctacggtgtcccgctaggaaagagaggtgcgtcaaacagcgacaagttccgcccacgtaaaagatgacgcttggtgtgtcagccgtccctgctgcccggttgcttctcttttgggggcggggtctagcaagagcaggtgtgggtttaggaggtgtgtgtttttgtttttcccaccctctctccccactacttgctctcacagtactcgctgagggtgaacaagaaaagacctgataaagattaaccagaagaaaacaaggagggaaacaaccgcagcctgtagcaagctctggaactcaggagtcgcgcgctaggggccggggccggggccggggcgtggtcggggcgggcccgggggcgggcccggggcggggctgcggttgcggtgcctgcgcccgcggcggcggaggcgcaggcggtggcgagtgggtgagtgaggaggcggcatcctggcgggtggctgtttggggttcggctgccgggaagaggcgcgggtagaagcgggggctctcctcagagctcgacgcatttttactttccctctcatttctctgaccgaagctgggtgtcgggctttcgcctctagcgactggtggaattgcctgcatccgggccccgggcttcccggcggcggcggcggcggcggcggcgcagggacaagggatggggatctggcctcttccttgctttcccgccctcagtacccgagctgtctccttcccggggacccgctgggagcgctgccgctgcgggctcgagaaaagggagcctcgggtactgagaggcctcgcctgggggaaggccggagggtgggcggcgcgcggcttctgcggaccaagtcggggttcgctaggaacccgagacggtccctgccggcgaggagatcatgcgggatgagatgggggtgtggagacgcctgcacaatttcagcccaagcttctagagagtggtgatgacttgcatatgagggcagcaatgcaagtcggtgtgctccccattctgtgggacatgacctggttgcttcacagctccgagatgacacagacttgcttaaaggaagtgactattgtgacttgggcatcacttgactgatggtaatcagttgtctaaagaagtgcacagattacatgtccgtgtgctcattgggtctatctggccgcgttgaacaccaccaggctttgtattcagaaacaggagggaggtcctgcactttcccaggaggggtggccctttcagatgcaatcgagattgttaggctctgggagagtagttgcctggttgtggcagttggtaaatttctattcaaacagttgccatgcaccagttgttcacaacaagggtacgtaatctgtctggcattacttctacttttgtacaaaggatcaaaaaaaaaaaagatactgttaagatatgatttttctcagactttgggaaacttttaacataatctgtgaatatcacagaaacaagactatcatataggggatattaataacctggagtcagaatacttgaaatacggtgtcatttgacacgggcattgttgtcaccacctctgccaaggcctgccactttaggaaaaccctgaatcagttggaaactgctacatgctgatagtacatctgaaacaagaacgagagtaattaccacattccagattgttcactaagccagcatttacctgctccaggaaaaaattacaagcaccttatgaagttgataaaatattttgtttggctatgttggcactccacaatttgctttcagagaaacaaagtaaaccaaggaggacttctgtttttcaagtctgccctcgggttctattctacgttaattagatagttcccaggaggactaggttagcctacctattgtctgagaaacttggaactgtgagaaatggccagatagtgatatgaacttcaccttccagtcttccctgatgttgaagattgagaaagtgttgtgaactttctggtactgtaaacagttcactgtccttgaagtggtcctgggcagctcctgttgtggaaagtggacggtttaggatcctgcttctctttgggctgggagaaaataaacagcatggttacaagtattgagagccaggttggagaaggtggcttacacctgtaatgccagagctttgggaggcggaggcaagaggatcacttgaagccaggagttcaagctcaacctgggcaacgtagaccctgtctctacaaaaaattaaaaacttagccgggcgtggtgatgtgcacctgtagtcctagctacttgggaggctgaggcaggagggtcatttgagcccaagagtttgaagttaccgagagctatgatcctgccagtgcattccagcctggatgacaaaacgagaccctgtctctaaaaaacaagaagtgagggctttatgattgtagaattttcactacaatagcagtggaccaaccacctttctaaataccaatcagggaagagatggttgattttttaacagacgtttaaagaaaaagcaaaacctcaaacttagcactctactaacagttttagcagatgttaattaatgtaatcatgtctgcatgtatgggattatttccagaaagtgtattgggaaacctctcatgaaccctgtgagcaagccaccgtctcactcaatttgaatcttggcttccctcaaaagactggctaatgtttggtaactctctggagtagacagcactacatgtacgtaagataggtacataaacaactattggttttgagctgatttttttcagctgcatttgcatgtatggatttttctcaccaaagacgatgacttcaagtattagtaaaataattgtacagctctcctgattatacttctctgtgacatttcatttcccaggctatttcttttggtaggatttaaaactaagcaattcagtatgatctttgtccttcattttctttcttattctttttgtttgtttgtttgtttgtttttttcttgaggcagagtctctctctgtcgcccaggctggagtgcagtggcgccatctcagctcattgcaacctctgccacctccgggttcaagagattctcctgcctcagcctcccgagtagctgggattacaggtgtccaccaccacacccggctaattttttgtatttttagtagaggtggggtttcaccatgttggccaggctggtcttgagctcctgacctcaggtgatccacctgcctcggcctaccaaagagctgggataacaggtgtgacccaccatgcccggcccattttttttttcttattctgttaggagtgagagtgtaactagcagtataatagttcaattttcacaacgtggtaaaagtttccctataattcaatcagattttgctccagggttcagttctgttttaggaaatacttttattttcagtttaatgatgaaatattagagttgtaatattgcctttatgattatccacctttttaacctaaaagaatgaaagaaaaatatgtttgcaatataattttatggttgtatgttaacttaattcattatgttggcctccagtttgctgttgttagttatgacagcagtagtgtcattaccatttcaattcagattacattcctatatttgatcattgtaaactgactgcttacattgtattaaaaacagtggatattttaaagaagctgtacggcttatatctagtgctgtctcttaagactattaaattgatacaacatatttaaaagtaaatattacctaaatgaatttttgaaattacaaatacacgtgttaaaactgtcgttgtgttcaaccatttctgtacatacttagagttaactgttttgccaggctctgtatgcctactcataatatgataaaagcactcatctaatgctctgtaaatagaagtcagtgctttccatcagactgaactctcttgacaagatgtggatgaaattctttaagtaaaattgtttactttgtcatacatttacagatcaaatgttagctcccaaagcaatcatatggcaaagataggtatatcatagtttgcctattagctgctttgtattgctattattataaatagacttcacagttttagacttgcttaggtgaaattgcaattctttttactttcagtcttagataacaagtcttcaattatagtacaatcacacattgcttaggaatgcatcattaggcgattttgtcattatgcaaacatcatagagtgtacttacacaaacctagatagtatagcctttatgtacctaggccgtatggtatagtctgttgctcctaggccacaaacctgtacaactgttactgtactgaatactatagacagttgtaacacagtggtaaatatttatctaaatatatgcaaacagagaaaaggtacagtaaaagtatggtataaaagataatggtatacctgtgtaggccacttaccacgaatggagcttgcaggactagaagttgctctgggtgagtcagtgagtgagtggtgaattaatgtgaaggcctagaacactgtacaccactgtagactataaacacagtacgctgaagctacaccaaatttatcttaacagtttttcttcaataaaaaattataactttttaactttgtaaactttttaattttttaacttttaaaatacttagcttgaaacacaaatacattgtatagctatacaaaaatattttttctttgtatccttattctagaagcttttttctattttctattttaaatttttttttttacttgttagtcgtttttgttaaaaactaaaacacacacactttcacctaggcatagacaggattaggatcatcagtatcactcccttccacctcactgccttccacctccacatcttgtcccactggaaggtttttaggggcaataacacacatgtagctgtcacctatgataacagtgctttctgttgaatacctcctgaaggacttgcctgaggctgttttacatttaacttaaaaaaaaaaaaagtagaaggagtgcactctaaaataacaataaaaggcatagtatagtgaatacataaaccagcaatgtagtagtttattatcaagtgttgtacactgtaataattgtatgtgctatactttaaataacttgcaaaatagtactaagaccttatgatggttacagtgtcactaaggcaatagcatattttcaggtccattgtaatctaatgggactaccatcatatatgcagtctaccattgactgaaacgttacatggcacataactgtatttgcaagaatgatttgttttacattaatatcacataggatgtacctttttagagtggtatgtttatgtggattaagatgtacaagttgagcaaggggaccaagagccctgggttctgtcttggatgtgagcgtttatgttcttctcctcatgtctgttttctcattaaattcaaaggcttgaacgggccctatttagcccttctgttttctacgtgttctaaataactaaagcttttaaattctagccatttagtgtagaactctctttgcagtgatgaaatgctgtattggtttcttggctagcatattaaatatttttatctttgtcttgatacttcaatgtcgttttaaacatcaggatcgggcttcagtattctcataaccagagagttcactgaggatacaggactgtttgcccattttttgttatggctccagacttgtggtatttccatgtctttttttttttttttttttttgaccttttagcggctttaaagtatttctgttgttaggtgttgtattacttttctaagattacttaacaaagcaccacaaactgagtggctttaaacaacagcaatttattctctcacaattctagaagctagaagtccgaaatcaaagtgttgacaggggcatgatcttcaagagagaagactctttccttgcctcttcctggcttctggtggttaccagcaatcctgagtgttcctttcttgccttgtagtttcaacaatccagtatctgccttttgtcttcacatggctgtctaccatttgtctctgtgtctccaaatctctctccttataaacacagcagttattggattaggccccactctaatccagtatgaccccattttaacatgattacacttatttctagataaggtcacattcacgtacaccaagggttaggaattgaacatatctttttgggggacacaattcaacccacaagtgtcagtctctagctgagcctttcccttcctgtttttctcctttttagttgctatgggttaggggccaaatctccagtcatactagaattgcacatggactggatatttgggaatactgcgggtctattctatgagctttagtatgtaacatttaatatcagtgtaaagaagcccttttttaagttatttctttgaatttctaaatgtatgccctgaatataagtaacaagttaccatgtcttgtaaaatgatcatatcaacaaacatttaatgtgcacctactgtgctagttgaatgtctttatcctgataggagataacaggattccacatctttgacttaagaggacaaaccaaatatgtctaaatcatttggggttttgatggatatctttaaattgctgaacctaatcattggtttcatatgtcattgtttagatatctccggagcatttggataatgtgacagttggaatgcagtgatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggtaagtgatttttcagcttattaatcatgttaacctatctgttgaaagcttattttctggtacatataaatcttatttttttaattatatgcagtgaacatcaaacaataaatgttatttattttgcatttaccctattagatacaaatacatctggtctgatacctgtcatcttcatattaactgtggaaggtacgaaatggtagctccacattatagatgaaaagctaaagcttagacaaataaagaaacttttagaccctggattcttcttgggagcctttgactctaataccttttgtttccctttcattgcacaattctgtcttttgcttactactatgtgtaagtataacagttcaaagtaatagtttcataagctgttggtcatgtagcctttggtctctttaacctctttgccaagttcccaggttcataaaatgaggaggttgaatggaatggttcccaagagaattccttttaatcttacagaaattattgttttcctaaatcctgtagttgaatatataatgctatttacatttcagtatagttttgatgtatctaaagaacacattgaattctccttcctgtgttccagtttgatactaacctgaaagtccattaagcattaccagttttaaaaggcttttgcccaatagtaaggaaaaataatatcttttaaaagaataattttttactatgtttgcaggcttacttccttttttctcacattatgaaactcttaaaatcaggagaatcttttaaacaacatcataatgtttaatttgaaaagtgcaagtcattcttttcctttttgaaactatgcagatgttacattgactgttttctgtgaagttatctttttttcactgcagaataaaggttgttttgattttattttgtattgtttatgagaacatgcatttgttgggttaatttcctacccctgcccccattttttccctaaagtagaaagtatttttcttgtgaactaaattactacacaagaacatgtctattgaaaaataagcaagtatcaaaatgttgtgggttgtttttttaaataaattttctcttgctcaggaaagacaagaaaatgtccagaagattatcttagaaggcacagagagaatggaagatcaggtatatgcaaattgcatactgtcaaatgtttttctcacagcatgtatctgtataaggttgatggctacatttgtcaaggccttggagacatacgaataagcctttaatggagcttttatggaggtgtacagaataaactggaggaagatttccatatcttaaacccaaagagttaaatcagtaaacaaaggaaaatagtaattgcatctacaaattaatatttgctccctttttttttctgtttgcccagaataaattttggataacttgttcatagtaaaaataaaaaaaattgtctctgatatgttctttaaggtactacttctcgaacctttccctagaagtagctgtaacagaaggagagcatatgtacccctgaggtatctgtctggggtgtaggcccaggtccacacaatatttcttctaagtcttatgttgtatcgttaagactcatgcaatttacattttattccataactattttagtattaaaatttgtcagtgatatttcttaccctctcctctaggaaaatgtgccatgtttatcccttggctttgaatgcccctcaggaacagacactaagagtttgagaagcatggttacaagggtgtggcttcccctgcggaaactaagtacagactatttcactgtaaagcagagaagttcttttgaaggagaatctccagtgaagaaagagttcttcacttttacttccatttcctcttgtgggtgaccctcaatgctccttgtaaaactccaatattttaaacatggctgttttgcctttctttgcttctttttagcatgaatgagacagatgatactttaaaaaagtaattaaaaaaaaaaacttgtgaaaatacatggccataatacagaacccaatacaatgatctcctttaccaaattgttatgtttgtacttttgtagatagctttccaattcagagacagttattctgtgtaaaggtctgacttaacaagaaaagatttccctttacccaaagaatcccagtccttatttgctggtcaataagcagggtccccaggaatggggtaactttcagcaccctctaacccactagttattagtagactaattaagtaaacttatcgcaagttgaggaaacttagaaccaactaaaattctgcttttactgggattttgttttttcaaaccagaaacctttacttaagttgactactattaatgaattttggtctctcttttaagtgctcttcttaaaaatgttatcttactgctgagaagttcaagtttgggaagtacaaggaggaatagaaacttaagagattttcttttagagcctcttctgtatttagccctgtaggatttttttttttttttttttttttggtgttgttgagcttcagtgaggctattcattcacttatactgataatgtctgagatactgtgaatgaaatactatgtatgcttaaacctaagaggaaatattttcccaaaattattcttcccgaaaaggaggagttgccttttgattgagttcttgcaaatctcacaacgactttattttgaacaatactgtttggggatgatgcattagtttgaaacaacttcagttgtagctgtcatctgataaaattgcttcacagggaaggaaatttaacacggatctagtcattattcttgttagattgaatgtgtgaattgtaattgtaaacaggcatgataattattactttaaaaactaaaaacagtgaatagttagttgtggaggttactaaaggatggtttttttttaaataaaactttcagcattatgcaaatgggcatatggcttaggataaaacttccagaagtagcatcacatttaaattctcaagcaacttaataatatggggctctgaaaaactggttaaggttactccaaaaatggccctgggtctgacaaagattctaacttaaagatgcttatgaagactttgagtaaaatcatttcataaaataagtgaggaaaaacaactagtattaaattcatcttaaataatgtatgatttaaaaaatatgtttagctaaaaatgcatagtcatttgacaatttcatttatatctcaaaaaatttacttaaccaagttggtcacaaaactgatgagactggtggtggtagtgaataaatgagggaccatccatatttgagacactttacatttgtgatgtgttatactgaattttcagtttgattctatagactacaaatttcaaaattacaatttcaagatgtaataagtagtaatatcttgaaatagctctaaagggaatttttctgttttattgattcttaaaatatatgtgctgattttgatttgcatttgggtagattatacttttatgagtatggaggttaggtattgattcaagttttccttacctatttggtaaggatttcaaagtctttttgtgcttggttttcctcatttttaaatatgaaatatattgatgacctttaacaaattttttttatctcaaattttaaaggagatcttttctaaaagaggcatgatgacttaatcattgcatgtaacagtaaacgataaaccaatgattccatactctctaaagaataaaagtgagctttagggccgggcatggtcagaaatttgacaccaacctggccaacatggcgaaaccccgtctctactaaaaatacaaaaatcagccgggcatggtggcggcacctatagtcccagctacttgggaggatgagacaggagagtcacttgaacctgggaggagaggttgcagtgagctgagatcacgccattgcactccagcctgagcaatgaaagcaaaactccatctcaaaaaaaaaaaaagaaaagaaagaataaaagtgagctttggattgcatataaatcctttagacatgtagtagacttgtttgatactgtgtttgaacaaattacgaagtattttcatcaaagaatgttattgtttgatgttatttttattttttattgcccagcttctctcatattacgtgattttcttcacttcatgtcactttattgtgcagggtcagagtattattccaatgcttactggagaagtgattcctgtaatggaactgctttcatctatgaaatcacacagtgttcctgaagaaatagatgtaagtttaaatgagagcaattatacactttatgagttttttggggttatagtattattatgtatattattaatattctaattttaatagtaaggactttgtcatacatactattcacatacagtattagccactttagcaaataagcacacacaaaatcctggattttatggcaaaacagaggcatttttgatcagtgatgacaaaattaaattcattttgtttatttcattacttttataattcctaaaagtgggaggatcccagctcttataggagcaattaatatttaatgtagtgtcttttgaaacaaaactgtgtgccaaagtagtaaccattaatggaagtttacttgtagtcacaaatttagtttccttaatcatttgttgaggacgttttgaatcacacactatgagtgttaagagatacctttaggaaactattcttgttgttttctgattttgtcatttaggttagtctcctgattctgacagctcagaagaggaagttgttcttgtaaaaattgtttaacctgcttgaccagctttcacatttgttcttctgaagtttatggtagtgcacagagattgttttttggggagtcttgattctcggaaatgaaggcagtgtgttatattgaatccagacttccgaaaacttgtatattaaaagtgttatttcaacactatgttacagccagactaatttttttattttttgatgcattttagatagctgatacagtactcaatgatgatgatattggtgacagctgtcatgaaggctttcttctcaagtaagaatttttcttttcataaaagctggatgaagcagataccatcttatgctcacctatgacaagatttggaagaaagaaaataacagactgtctacttagattgttctagggacattacgtatttgaactgttgcttaaatttgtgttatttttcactcattatatttctatatatatttggtgttattccatttgctatttaaagaaaccgagtttccatcccagacaagaaatcatggccccttgcttgattctggtttcttgttttacttctcattaaagctaacagaatcctttcatattaagttgtactgtagatgaacttaagttatttaggcgtagaacaaaattattcatatttatactgatctttttccatccagcagtggagtttagtacttaagagtttgtgcccttaaaccagactccctggattaatgctgtgtacccgtgggcaaggtgcctgaattctctatacacctatttcctcatctgtaaaatggcaataatagtaatagtacctaatgtgtagggttgttataagcattgagtaagataaataatataaagcacttagaacagtgcctggaacataaaaacacttaataatagctcatagctaacatttcctatttacatttcttctagaaatagccagtatttgttgagtgcctacatgttagttcctttactagttgctttacatgtattatcttatattctgttttaaagtttcttcacagttacagattttcatgaaattttacttttaataaaagagaagtaaaagtataaagtattcacttttatgttcacagtcttttcctttaggctcatgatggagtatcagaggcatgagtgtgtttaacctaagagccttaatggcttgaatcagaagcactttagtcctgtatctgttcagtgtcagcctttcatacatcattttaaatcccatttgactttaagtaagtcacttaatctctctacatgtcaatttcttcagctataaaatgatggtatttcaataaataaatacattaattaaatgatattatactgactaattgggctgttttaaggctcaataagaaaatttctgtgaaaggtctctagaaaatgtaggttcctatacaaataaaagataacattgtgcttatagcttcggtgtttatcatataaagctattctgagttatttgaagagctcacctacttttttttgtttttagtttgttaaattgttttataggcaatgtttttaatctgttttctttaacttacagtgccatcagctcacacttgcaaacctgtggctgttccgttgtagtaggtagcagtgcagagaaagtaaataaggtagtttattttataatctagcaaatgatttgactctttaagactgatgatatatcatggattgtcatttaaatggtaggttgcaattaaaatgatctagtagtataaggaggcaatgtaatctcatcaaattgctaagacaccttgtggcaacagtgagtttgaaataaactgagtaagaatcatttatcagtttattttgatagctcggaaataccagtgtcagtagtgtataaatggttttgagaatatattaaaatcagatatataaaaaaaattactcttctatttcccaatgttatctttaacaaatctgaagatagtcatgtacttttggtagtagttccaaagaaatgttatttgtttattcatcttgatttcattgtcttcgctttccttctaaatctgtcccttctagggagctattgggattaagtggtcattgattattatactttattcagtaatgtttctgaccctttccttcagtgctacttgagttaattaaggattaatgaacagttacatttccaagcattagctaataaactaaaggattttgcacttttcttcactgaccattagttagaaagagttcagagataagtatgtgtatctttcaatttcagcaaacctaattttttaaaaaaagttttacataggaaatatgttggaaatgatactttacaaagatattcataatttttttttgtaatcagctactttgtatatttacatgagccttaatttatatttctcatataaccatttatgagagcttagtatacctgtgtcattatattgcatctacgaactagtgaccttattccttctgttacctcaaacaggtggctttccatctgtgatctccaaagccttaggttgcacagagtgactgccgagctgctttatgaagggagaaaggctccatagttggagtgtttttttttttttttttaaacatttttcccatcctccatcctcttgagggagaatagcttaccttttatcttgttttaatttgagaaagaagttgccaccactctaggttgaaaaccactcctttaacataataactgtggatatggtttgaatttcaagatagttacatgcctttttatttttcctaatagagctgtaggtcaaatattattagaatcagatttctaaatcccacccaatgacctgcttattttaaatcaaattcaataattaattctcttctttttggaggatctggacattctttgatatttcttacaacgaatttcatgtgtagacccactaaacagaagctataaaagttgcatggtcaaataagtctgagaaagtctgcagatgatataattcacctgaagagtcacagtatgtagccaaatgttaaaggttttgagatgccatacagtaaatttaccaagcattttctaaatttatttgaccacagaatccctattttaagcaacaactgttacatcccatggattccaggtgactaaagaatacttatttcttaggatatgttttattgataataacaattaaaatttcagatatctttcataagcaaatcagtggtctttttacttcatgttttaatgctaaaatattttcttttatagatagtcagaacattatgcctttttctgactccagcagagagaaaatgctccaggttatgtgaagcagaatcatcatttaaatatgagtcagggctctttgtacaaggcctgctaaaggtatagtttctagttatcacaagtgaaaccacttttctaaaatcatttttgagactctttatagacaaatcttaaatattagcatttaatgtatctcatattgacatgcccagagactgacttcctttacacagttctgcacatagactatatgtcttatggatttatagttagtatcatcagtgaaacaccatagaataccctttgtgttccaggtgggtccctgttcctacatgtctagcctcaggacttttttttttttaacacatgcttaaatcaggttgcacatcaaaaataagatcatttctttttaactaaatagatttgaattttattgaaaaaaaattttaaacatctttaagaagcttataggatttaagcaattcctatgtatgtgtactaaaatatatatatttctatatataatatatattagaaaaaaattgtatttttcttttatttgagtctactgtcaaggagcaaaacagagaaatgtaaattagcaattatttataatacttaaagggaagaaagttgttcaccttgttgaatctattattgttatttcaattatagtcccaagacgtgaagaaatagctttcctaatggttatgtgattgtctcatagtgactactttcttgaggatgtagccacggcaaaatgaaataaaaaaatttaaaaattgttgcaaatacaagttatattaggcttttgtgcattttcaataatgtgctgctatgaactcagaatgatagtatttaaatatagaaactagttaaaggaaacgtagtttctatttgagttatacatatctgtaaattagaacttctcctgttaaaggcataataaagtgcttaatacttttgtttcctcagcaccctctcatttaattatataattttagttctgaaagggacctataccagatgcctagaggaaatttcaaaactatgatctaatgaaaaaatatttaatagttctccatgcaaatacaaatcatatagttttccagaaaatacctttgacattatacaaagatgattatcacagcattataatagtaaaaaaatggaaatagcctctttcttctgttctgttcatagcacagtgcctcatacgcagtaggttattattacatggtaactggctaccccaactgattaggaaagaagtaaatttgttttataaaaatacatactcattgaggtgcatagaataattaagaaattaaaagacacttgtaattttgaatccagtgaatacccactgttaatatttggtatatctctttctagtctttttttcccttttgcatgtattttctttaagactcccacccccactggatcatctctgcatgttctaatctgcttttttcacagcagattctaagcctctttgaatatcaacacaaacttcaacaacttcatctatagatgccaaataataaattcatttttatttacttaaccacttcctttggatgcttaggtcattctgatgttttgctattgaaaccaatgctatactgaacacttctgtcactaaaactttgcacacactcatgaatagcttcttaggataaatttttagagatggatttgctaaatcagagaccattttttaaaattaaaaaacaattattcatatcgtttggcatgtaagacagtaaattttccttttattttgacaggattcaactggaagctttgtgctgcctttccggcaagtcatgtatgctccatatcccaccacacacatagatgtggatgtcaatactgtgaagcagatgccaccctgtcatgaacatatttataatcagcgtagatacatgagatccgagctgacagccttctggagagccacttcagaagaagacatggctcaggatacgatcatctacactgacgaaagctttactcctgatttgtacgtaatgctctgcctgctggtactgtagtcaagcaatatgaaattgtgtcttttacgaataaaaacaaaacagaagttgcatttaaaaagaaagaaatattaccagcagaattatgcttgaagaaacatttaatcaagcatttttttcttaaatgttcttctttttccatacaattgtgtttaccctaaaataggtaagattaacccttaaagtaaatatttaactatttgtttaataaatatatattgagctcctaggcactgttctaggtaccgggcttaatagtggccaaccagacagccccagccccagcccctacattgtgtatagtctattatgtaacagttattgaatggacttattaacaaaaccaaagaagtaattctaagtcttttttttcttgacatatgaatataaaatacagcaaaactgttaaaatatattaatggaacatttttttactttgcattttatattgttattcacttcttatttttttttaaaaaaaaaagcctgaacagtaaattcaaaaggaaaagtaatgataattaattgttgagcatggacccaacttgaaaaaaaaaatgatgatgataaatctataatcctaaaaccctaagtaaacacttaaaagatgttctgaaatcaggaaaagaattatagtatacttttgtgtttctcttttatcagttgaaaaaaggcacagtagctcatgcctgtaagaacagagctttgggagtgcaaggcaggcggatcacttgaggccaggagttccagaccagcctgggcaacatagtgaaaccccatctctacaaaaaataaaaaagaattattggaatgtgtttctgtgtgcctgtaatcctagctattccgaaagctgaggcaggaggatcttttgagcccaggagtttgaggttacagggagttatgatgtgccagtgtactccagcctggggaacaccgagactctgtcttatttaaaaaaaaaaaaaaaaaaatgcttgcaataatgcctggcacatagaaggtaacagtaagtgttaactgtaataacccaggtctaagtgtgtaaggcaatagaaaaattggggcaaataagcctgacctatgtatctacagaatcagtttgagcttaggtaacagacctgtggagcaccagtaattacacagtaagtgttaaccaaaagcatagaataggaatatcttgttcaagggacccccagccttatacatctcaaggtgcagaaagatgacttaatataggacccattttttcctagttctccagagtttttattggttcttgagaaagtagtaggggaatgttttagaaaatgaattggtccaactgaaattacatgtcagtaagtttttatatattggtaaattttagtagacatgtagaagttttctaattaatctgtgccttgaaacattttcttttttcctaaagtgcttagtattttttccgttttttgattggttacttgggagcttttttgaggaaatttagtgaactgcagaatgggtttgcaaccatttggtatttttgttttgttttttagaggatgtatgtgtattttaacatttcttaatcatttttagccagctatgtttgttttgctgatttgacaaactacagttagacagctattctcattttgctgatcatgacaaaataatatcctgaatttttaaattttgcatccagctctaaattttctaaacataaaattgtccaaaaaatagtattttcagccactagattgtgtgttaagtctattgtcacagagtcattttacttttaagtatatgtttttacatgttaattatgtttgttatttttaattttaactttttaaaataattccagtcactgccaatacatgaaaaattggtcactggaattttttttttgacttttattttaggttcatgtgtacatgtgcaggtgtgttatacaggtaaattgcgtgtcatgagggtttggtgtacaggtgatttcattacccaggtaataagcatagtacccaataggtagttttttgatcctcacccttctcccaccctcaagtaggccctggtgttgctgtttccttctttgtgtccatgtatactcagtgtttagctcccacttagaagtgagaacatgcggtagttggttttctgttcctggattagttcacttaggataatgacctctagctccatctggtttttatggctgcatagtattccatggtgtatatgtatcacattttctttatccagtctaccattgataggcatttaggttgattccctgtctttgttatcatgaatagtgctgtgatgaacatacacatgcatgtgtctttatggtagaaaaatttgtattcctttaggtacatatagaataatggggttgctagggtgaatggtagttctattttcagttatttgagaaatcttcaaactgcttttcataatagctaaactaatttacagtcccgccagcagtgtataagtgttcccttttctccacaaccttgccaacatctgtgattttttgactttttaataatagccattcctagagaattgatttgcaattctctattagtgatattaagcattttttcatatgctttttagctgtctgtatatattcttctgaaaaattttcatgtcctttgcccagtttgtagtggggtgggttgttttttgcttgttaattagttttaagttccttccagattctgcatatccctttgttggatacatggtttgcagatatttttctcccattgtgtaggttgtcttttactctgttgatagtttcttttgccatgcaggagctcgttaggtcccatttgtgtttgtttttgttgcagttgcttttggcgtcttcatcataaaatctgtgccagggcctatgtccagaatggtatttcctaggttgtcttccagggtttttacaattttagattttacgtttatgtctttaatccatcttgagttgatttttgtatatggcacaaggaaggggtccagtttcactccaattcctatggctagcaattatcccagcaccatttattgaatacggagtcctttccccattgcttgttttttgtcaactttgttgaagatcagatggttgtaagtgtgtggctttatttcttggctctctattctccattggtctatgtgtctgtttttataacagtaccctgctgttcaggttcctatagccttttagtataaaatcggctaatgtgatgcctccagctttgttctttttgcttaggattgctttggctatttgggctcctttttgggtccatattaattttaaaacagttttttctggttttgtgaaggatatcattggtagtttataggaatagcattgaatctgtagattgctttgggcagtatggccattttaacaatattaattcttcctatctatgaatatggaatgtttttccatgtgtttgtgtcatctctttatacctgatgtataaagaaaagctggtattattcctactcaatctgttccaaaaaattgaggaggaggaactcttccctaatgaggccagcatcattctgataccaaaacctggcagagacacaacagaaaaaagaaaacttcaggccaatatccttgatgaatatagatgcaaaaatcctcaacaaaatactagcaaaccaaatccagcagcacatcaaaaagctgatctactttgatcaagtaggctttatccctgggatgcaaggttggttcaacatacacaaatcaataagtgtgattcatcacataaacagagctaaaaacaaaaaccacaagattatctcaataggtagagaaaaggttgtcaataaaatttaacatcctccatgttaaaaaccttcagtaggtcaggtgtagtgactcacacctgtaatcccagcactttgggaggccaaggcgggcatatctcttaagcccaggagttcaagacgagcctaggcagcatggtgaaaccccatctctacaaaaaaaaaaaaaaaaaaaaattagcttggtatggtgacatgcacctatagtcccagctattcaggaggttgaggtgggaggattgtttgagcccgggaggcagaggttggcagcgagctgagatcatgccaccgcactccagcctgggcaacggagtgagaccctgtctcaaaaaagaaaaatcacaaacaatcctaaacaaactaggcattgaaggaacatgcctcaaaaaaataagaaccatctatgacagacccatagccaatatcttaccaaatgggcaaaagctggaagtattctccttgagaaccgtaacaagacaaggatgtccactctcaccactccttttcagcatagttctggaagtcctagccagagcaatcaggaaagagaaagaaagaaagacattcagataggaagagaagaagtcaaactatttctgtttgcaggcagtataattctgtacctagaaaatctcatagtctctgcccagaaactcctaaatctgttaaaaatttcagcaaagttttggcattctctatactccaacaccttccaaagtgagagcaaaatcaagaacacagtcccattcacaatagccgcaaaacgaataaaatacctaggaatccagctaaccagggaggtgaaagatctctatgagaattacaaaacactgctgaaagaaatcagagatgacacaaacaaatggaaatgttcttttttaacaccttgctttatctaattcacttatgatgaagatactcattcagtggaacaggtataataagtccactcgattaaatataagccttattctctttccagagcccaagaaggggcactatcagtgcccagtcaataatgacgaaatgctaatatttttcccctttacggtttctttcttctgtagtgtggtacactcgtttcttaagataaggaaacttgaactaccttcctgtttgcttctacacatacccattctctttttttgccactctggtcaggtataggatgatccctaccactttcagttaaaaactcctcctcttactaaatgttctcttaccctctggcctgagtagaacctagggaaaatggaagagaaaaagatgaaagggaggtggggcctgggaagggaataagtagtcctgtttgtttgtgtgtttgctttagcacctgctatatcctaggtgctgtgttaggcacacattattttaagtggccattatattactactactcactctggtcgttgccaaggtaggtagtactttcttggatagttggttcatgttacttacagatggtgggcttgttgaggcaaacccagtggataatcatcggagtgtgttctctaatctcactcaaatttttcttcacattttttggtttgttttggtttttgatggtagtggcttatttttgttgctggtttgttttttgtttttttttgagatggcaagaattggtagttttatttattaattgcctaagggtctctactttttttaaaagatgagagtagtaaaatagattgatagatacatacatacccttactggggactgcttatattctttagagaaaaaattacatattagcctgacaaacaccagtaaaatgtaaatatatccttgagtaaataaatgaatgtatattttgtgtctccaaatatatatatctatattcttacaaatgtgtttatatgtaatatcaatttataagaacttaaaatgttggctcaagtgagggattgtggaaggtagcattatatggccatttcaacatttgaacttttttcttttcttcattttcttcttttcttcaggaatatttttcaagatgtcttacacagagacactctagtgaaagccttcctggatcaggtaaatgttgaacttgagattgtcagagtgaatgatatgacatgttttcttttttaatatatcctacaatgcctgttctatatatttatattcccctggatcatgccccagagttctgctcagcaattgcagttaagttagttacactacagttctcagaagagtctgtgagggcatgtcaagtgcatcattacattggttgcctcttgtcctagatttatgcttcgggaattcagacctttgtttacaatataataaatattattgctatcttttaaagatataataataagatataaagttgaccacaactactgttttttgaaacatagaattcctggtttacatgtatcaaagtgaaatctgacttagcttttacagatataatatatacatatatatatcctgcaatgcttgtactatatatgtagtacaagtatatatatatgtttgtgtgtgtatatatatatagtacgagcatatatacatattaccagcattgtaggatatatatatgtttatatattaaaaaaaagttataaacttaaaaccctattatgttatgtagagtatatgttatatatgatatgtaaaatatataacatatactctatgatagagtgtaatatattttttatatatattttaacatttataaaatgatagaattaagaattgagtcctaatctgttttattaggtgctttttgtagtgtctggtctttctaaagtgtctaaatgatttttccttttgacttattaatggggaagagcctgtatattaacaattaagagtgcagcattccatacgtcaaacaacaaacattttaattcaagcattaacctataacaagtaagttttttttttttttttgagaaagggaggttgtttatttgcctgaaatgactcaaaaatatttttgaaacatagtgtacttatttaaataacatctttattgtttcattcttttaaaaaatatctacttaattacacagttgaaggaaatcgtagattatatggaacttatttcttaatatattacagtttgttataataacattctggggatcaggccaggaaactgtgtcatagataaagctttgaaataatgagatccttatgtttactagaaattttggattgagatctatgaggtctgtgacatattgcgaagttcaaggaaaattcgtaggcctggaatttcatgcttctcaagctgacataaaatccctcccactctccacctcatcatatgcacacattctactcctacccacccactccaccccctgcaaaagtacaggtatatgaatgtctcaaaaccataggctcatcttctaggagcttcaatgttatttgaagatttgggcagaaaaaattaagtaatacgaaataacttatgtatgagttttaaaagtgaagtaaacatggatgtattctgaagtagaatgcaaaatttgaatgcatttttaaagataaattagaaaacttctaaaaactgtcagattgtctgggcctggtggcttatgcctgtaatcccagcactttgggagtccgaggtgggtggatcacaaggtcaggagatcgagaccatcctgccaacatggtgaaaccccgtctctactaagtatacaaaaattagctgggcgtggcagcgtgtgcctgtaatcccagctacctgggaggctgaggcaggagaatcgcttgaacccaggaggtgtaggttgcagtgagtcaagatcgcgccactgcactttagcctggtgacagagctagactccgtctcaaaaaaaaaaaaaaatatcagattgttcctacacctagtgcttctataccacactcctgttagggggcatcagtggaaatggttaaggagatgtttagtgtgtattgtctgccaagcactgtcaacactgtcatagaaacttctgtacgagtagaatgtgagcaaattatgtgttgaaatggttcctctccctgcaggtctttcagctgaaacctggcttatctctcagaagtactttccttgcacagtttctacttgtccttcacagaaaagccttgacactaataaaatatatagaagacgatacgtgagtaaaactcctacacggaagaaaaacctttgtacattgtttttttgttttgtttcctttgtacattttctatatcataatttttgcgcttcttttttttttttttttttttttttttccattatttttaggcagaagggaaaaaagccctttaaatctcttcggaacctgaagatagaccttgatttaacagcagagggcgatcttaacataataatggctctggctgagaaaattaaaccaggcctacactcttttatctttggaagacctttctacactagtgtgcaagaacgagatgttctaatgactttttaaatgtgtaacttaataagcctattccatcacaatcatgatcgctggtaaagtagctcagtggtgtggggaaacgttcccctggatcatactccagaattctgctctcagcaattgcagttaagtaagttacactacagttctcacaagagcctgtgaggggatgtcaggtgcatcattacattgggtgtctcttttcctagatttatgcttttgggatacagacctatgtttacaatataataaatattattgctatcttttaaagatataataataggatgtaaacttgaccacaactactgtttttttgaaatacatgattcatggtttacatgtgtcaaggtgaaatctgagttggcttttacagatagttgactttctatcttttggcattctttggtgtgtagaattactgtaatacttctgcaatcaactgaaaactagagcctttaaatgatttcaattccacagaaagaaagtgagcttgaacataggatgagctttagaaagaaaattgatcaagcagatgtttaattggaattgattattagatcctactttgtggatttagtccctgggattcagtctgtagaaatgtctaatagttctctatagtccttgttcctggtgaaccacagttagggtgttttgtttattttattgttcttgctattgttgatattctatgtagttgagctctgtaaaaggaaattgtattttatgttttagtaattgttgccaactttttaaattaattttcattatttttgagccaaattgaaatgtgcacctcctgtgccttttttctccttagaaaatctaattacttggaacaagttcagatttcactggtcagtcattttcatcttgttttcttcttgctaagtcttaccatgtacctgctttggcaatcattgcaactctgagattataaaatgccttagagaatatactaactaataagatctttttttcagaaacagaaaatagttccttgagtacttccttcttgcatttctgcctatgtttttgaagttgttgctgtttgcctgcaataggctataaggaatagcaggagaaattttactgaagtgctgttttcctaggtgctactttggcagagctaagttatcttttgttttcttaatgcgtttggaccattttgctggctataaaataactgattaatataattctaacacaatgttgacattgtagttacacaaacacaaataaatattttatttaaaattctggaagtaatataaaagggaaaatatatttataagaaagggataaaggtaatagagcccttctgccccccacccaccaaatttacacaacaaaatgacatgttcgaatgtgaaaggtcataatagctttcccatcatgaatcagaaagatgtggacagcttgatgttttagacaaccactgaactagatgactgttgtactgtagctcagtcatttaaaaaatatataaatactaccttgtagtgtcccatactgtgttttttacatggtagattcttatttaagtgctaactggttattttctttggctggtttattgtactgttatacagaatgtaagttgtacagtgaaataagttattaaagcatgtgtaaacattgttatatatcttttctcctaaatggagaattttgaataaaatatatttgaaattttgcctctttcagttgttcattcagaaaaaaatactatgatatttgaagactgatcagcttctgttcagctgacagtcatgctggatctaaactttttttaaaattaattttgtcttttcaaagaaaaaatatttaaagaagctttataatataatcttatgttaaaaaaactttctgcttaactctctggatttcattttgatttttcaaattatatattaatatttcaaatgtaaaatactatttagataaattgtttttaaacattcttattattataatattaatataacctaaactgaagttattcatcccaggtatctaatacatgtatccaaagtaaaaatccaaggaatctgaacactttcatctgcaaagctaggaataggtttgacattttcactccaagaaaaagtttttttttgaaaatagaatagttgggatgagaggtttctttaaaagaagactaactgatcacattactatgattctcaaagaagaaaccaaaacttcatataatactataaagtaaatataaaatagttccttctatagtatatttctataatgctacagtttaaacagatcactcttatataatactattttgattttgatgtagaattgcacaaattgatatttctcctatgatctgcagggtatagcttaaagtaacaaaaacagtcaaccacctccatttaacacacagtaacactatgggactagttttattacttccattttacaaatgaggaaactaaagcttaaagatgtgtaatacaccgcccaaggtcacacagctggtaaaggtggatttcatcccagacagttacagtcattgccatgggcacagctcctaacttagtaactccatgtaactggtactcagtgtagctgaattgaaaggagagtaaggaagcaggttttacaggtctacttgcactattcagagcccgagtgtgaatccctgctgtgctgcttggagaagttacttaacctatgcaaggttcattttgtaaatattggaaatggagtgataatacgtacttcaccagaggatttaatgagaccttatacgatccttagttcagtacctgactagtgcttcataaatgctttttcatccaatctgacaatctccagcttgtaattggggcatttagaacatttaatatgattattggcatggtaggttaaagctgtcatcttgctgttttctatttgttctttttgttttctccttacttttggatttttttattctactatgtcttttctattgtcttattaactatactctttgatttattttagtggttgttttagggttatacctctttctaatttaccagtttataaccagtttatatactacttgacatatagcttaagaaacttactgttgttgtctttttgctgttatggtcttaacgtttttatttctacaaacattataaactccacactttattgttttttaattttacttatacagtcaattatcttttaaagatatttaaatataaacattcaaaacaccccaat

SEQ ID NO: 3 - DNA, 20 bp, Artificial sequence
  Primer
gggtctagcaagagcaggtg

SEQ ID NO: 4 - DNA, 21 bp, Artificial sequence
  Primer
gtcttggcaacagctggagat

SEQ ID NO: 5 - DNA, 24 bp, Artificial sequence
  Probe
tgatgtcgactctttgcccaccgc

SEQ ID NO: 6 - DNA, 18 bp, Artificial sequence
  Synthetic oligonucleotide
gcccctagcgcgcgactc

(19) United States
(12) Patent Application Publication
(10) Pub. No.: US 2019/0142856 A1
(43) Pub. Date: May 16, 2019

(54) METHODS FOR REDUCING C9ORF72 EXPRESSION

(71) Applicants: Ionis Pharmaceuticals, Inc., Carlsbad, CA (US); Ludwing Institute for Cancer Research, Zurich (CH)

(72) Inventors: C. Frank Bennett, Carlsbad, CA (US); Frank Rigo, Carlsbad, CA (US); Don W. Cleveland, La Jolla, CA (US); Jie Jiang, San Diego, CA (US); Qiang Zhu, San Diego, CA (US); Clotilde Lagier-Tourene, Winchester, MA (US)

(73) Assignees: Ionis Pharmaceuticals, Inc., Carlsbad, CA (US); Ludwig Institute for Cancer Research, Zurich (CH)

(21) Appl. No.: 16/092,183

(22) PCT Filed: Apr. 13, 2017
(86) PCT No.: PCT/US2017/027355

§ 371 Date: Oct. 8, 2018

Related U.S. Application Data
(60) Provisional application No. 62/322,146, filed on Apr. 13, 2016.

(51) Int. Cl. A61K 31/712; A61P 25/28; A61K 31/7105; A61K 31/7115

(52) U.S. Cl. CPC A61K 31/712; A61K 31/7115; A61K 31/7105; A61P 25/28

(57) ABSTRACT

Provided are methods for reducing the amount or activity of C90RF72 RNA, and in certain instances of reducing the amount of C90RF72 protein, in an animal. Such methods are useful to prevent or ameliorate at least one symptom of a neurodegenerative disease. Such symptoms include anxiety, reduced spatial learning, and memory loss. Such neurodegenerative diseases include amyotrophic lateral sclerosis (ALS), frontotemporal dementia (FTD), corticalbasal degeneration syndrome (CBD), atypical Parkinsonian syndrome, and olivopontocerellar degeneration (OPCD).

### STATEMENT OF GOVERNMENT SUPPORT

This invention was made with government support under R01-NS088578, R01-NS087227, and P50-AG005131 awarded by National Institute of Health. The government has certain rights in the invention.

### SEQUENCE LISTING

The present application is being filed along with a Sequence Listing in electronic format. The Sequence Listing is provided as a file entitled BIOL0288WOSEQ_ST25.txt, created on Apr. 12, 2017, which is 44 KB in size. The information in the electronic format of the sequence listing is incorporated herein by reference in its entirety.

### FIELD

Provided are methods for reducing the amount or activity of C9ORF72 RNA, and in certain instances of reducing the amount of C9ORF72 protein, in an animal. Such methods are useful to prevent or ameliorate at least one symptom of a neurodegenerative disease. Such symptoms include anxiety, reduced spatial learning, and memory loss. Such neurodegenerative diseases include amyotrophic lateral sclerosis (ALS), frontotemporal dementia (FTD), corticalbasal degeneration syndrome (CBD), atypical Parkinsonian syndrome, and olivopontocerellar degeneration (OPCD).

### BACKGROUND

Amyotrophic lateral sclerosis (ALS) is a fatal neurodegenerative disease characterized clinically by progressive paralysis leading to death from respiratory failure, typically within two to three years of symptom onset (Rowland and Shneider, N. Engl. J. Med., 2001, 344, 1688-1700). ALS is the third most common neurodegenerative disease in the Western world (Hirtz et al., Neurology, 2007, 68, 326-337), and there are currently no effective therapies. Approximately 10% of cases are familial in nature, whereas the bulk of patients diagnosed with the disease are classified as sporadic as they appear to occur randomly throughout the population (Chio et al., Neurology, 2008, 70, 533-537). There is growing recognition, based on clinical, genetic, and epidemiological data, that ALS and frontotemporal dementia (FTD) represent an overlapping continuum of disease, characterized pathologically by the presence of TDP-43 positive inclusions throughout the central nervous system (Lillo and Hodges, J. Clin. Neurosci., 2009, 16, 1131-1135; Neumann et al., Science, 2006, 314, 130-133).

To date, a number of genes have been discovered as causative for classical familial ALS, for example, SOD1, TARDBP, FUS, OPTN, and VCP (Johnson et al., Neuron, 2010, 68, 857-864; Kwiatkowski et al., Science, 2009, 323, 1205-1208; Maruyama et al., Nature, 2010, 465, 223-226; Rosen et al., Nature, 1993, 362, 59-62; Sreedharan et al., Science, 2008, 319, 1668-1672; Vance et al., Brain, 2009, 129, 868-876). Recently, linkage analysis of kindreds involving multiple cases of ALS, FTD, and ALS-FTD had suggested that there was an important locus for the disease on the short arm of chromosome 9 (Boxer et al., J. Neurol. Neurosurg. Psychiatry, 2011, 82, 196-203; Morita et al., Neurology, 2006, 66, 839-844; Pearson et al. J. Nerol., 2011, 258, 647-655; Vance et al., Brain, 2006, 129, 868-876). The chromosome 9p21ALS-FTD locus in the last major autosomal-dominant gene whose mutation is causative of ALS. The ALS-FTD causing mutation is a large hexanucleotide (GGGGCC) repeat expansion in the first intron of the C9ORF72 gene (Renton et al., Neuron, 2011, 72, 257-268; DeJesus-Hernandez et al., Neuron, 2011, 72, 245-256). A founder haplotype, covering the C9ORF72 gene, is present in the majority of cases linked to this region (Renton et al., Neuron, 2011, 72, 257-268). This locus on chromosome 9p21 accounts for nearly half of familial ALS and nearly one-quarter of all ALS cases in a cohort of 405 Finnish patients (Laaksovirta et al, Lancet Neurol., 2010, 9, 978-985).

Currently there is a lack of acceptable options for treating neurodegenerative diseases such as amyotrophic lateral sclerosis (ALS), frontotemporal dementia (FTD), corticalbasal degeneration syndrome (CBD), atypical Parkinsonian syndrome, and olivopontocerellar degeneration (OPCD). It is therefore an object herein to provide methods for the treatment of such diseases.

### SUMMARY OF THE INVENTION

Provided are methods for reducing the amount or activity of C9ORF72 RNA, and in certain instances of reducing the amount of C9ORF72 protein, in an animal. In certain embodiments, the animal has a neurodegenerative disease. In certain embodiments, the neurodegenerative disease is amyotrophic lateral sclerosis (ALS), frontotemporal dementia (FTD), corticalbasal degeneration syndrome (CBD), atypical Parkinsonian syndrome, or olivopontocerellar degeneration (OPCD). In certain embodiments, compounds useful for reducing the amount or activity of C9ORF72 RNA are oligomeric compounds. In certain embodiments, the oligomeric compound comprises a modified oligonucleotide.

Also provided are methods useful for ameliorating or preventing at least one symptom of a neurodegenerative disease. In certain embodiments, the neurodegenerative disease is amyotrophic lateral sclerosis (ALS), frontotemporal dementia (FTD), corticalbasal degeneration syndrome (CBD), atypical Parkinsonian syndrome, or olivopontocerellar degeneration (OPCD). In certain embodiments symptoms include anxiety, reduced spatial learning, and memory loss. In certain embodiments, amelioration of these symptoms results in reduced anxiety, improved spatial learning, or improved memory.

### DETAILED DESCRIPTION OF THE INVENTION

It is to be understood that both the foregoing general description and the following detailed description are exemplary and explanatory only and are not restrictive. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated-by-reference for the portions of the document discussed herein, as well as in their entirety.

### Definitions

Unless specific definitions are provided, the nomenclature used in connection with, and the procedures and techniques of, analytical chemistry, synthetic organic chemistry, and medicinal and pharmaceutical chemistry described herein are those well known and commonly used in the art. Where permitted, all patents, applications, published applications and other publications and other data referred to throughout in the disclosure are incorporated by reference herein in their entirety.

Unless otherwise indicated, the following terms have the following meanings:

### Definitions

“Administering” means providing a pharmaceutical agent to an animal. “Administered prior to the detection of the at least one symptom” is prophylactic administration and means providing the pharmaceutical agent to an animal before a symptom of a neurodegenerative disease is apparent through diagnosis. Such diagnosis may be accomplished by, for example, clinical evaluation and genetic testing.

“Animal” means a human or non-human animal.

“Antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound.

“Ameliorate” or “amelioration” in reference to a treatment means improvement in at least one symptom relative to the same symptom in the absence of the treatment. In certain embodiments, amelioration is the reduction in the severity or frequency of a symptom or the delayed onset or slowing of progression in the severity or frequency of a symptom. In certain embodiments, symptoms are anxiety, reduced spatial learning, and memory loss.

“Bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the bicyclic sugar moiety does not comprise a furanosyl moiety.

“Complementary” in reference to an oligonucleotide means that at least 70% of the nucleobases of the oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. Complementary nucleobases means nucleobases that are capable of forming hydrogen bonds with one another. Complementary nucleobase pairs include, but unless otherwise specific are not limited to, adenine (A) and thymine (T), adenine (A) and uracil (U), cytosine (C) and guanine (G), 5-methyl cytosine (mC) and guanine (G). Complementary oligonucleotides and/or nucleic acids need not have nucleobase complementarity at each nucleoside. Rather, some mismatches are tolerated. As used herein, “fully complementary” or “100% complementary” in reference to oligonucleotides means that oligonucleotides are complementary to another oligonucleotide or nucleic acid at each nucleoside of the oligonucleotide.

“Conjugate group” means a group of atoms that is directly or indirectly attached to an oligonucleotide. Conjugate groups include a conjugate moiety and a conjugate linker that attaches the conjugate moiety to the oligonucleotide.

“Contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

“Gapmer” means a modified oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.”

“Internucleoside linkage” is the covalent linkage between adjacent nucleosides in an oligonucleotide. As used herein “modified internucleoside linkage” means any internucleoside linkage other than a phosphodiester internucleoside linkage. “Phosphorothioate linkage” means a modified internucleoside linkage in which one of the non-bridging oxygen atoms of a phosphodiester internucleoside linkage is replaced with a sulfur atom.

“MOE” means methoxyethyl. “2′-MOE” means a —OCH₂CH₂OCH₃ group at the 2′ position of a furanosyl ring.

“Neurodegenerative disease” means a condition marked by progressive loss of structure or function of neurons, including death of neurons. In certain embodiments, neurodegenerative disease is any of amyotrophic lateral sclerosis (ALS), frontotemporal dementia (FTD), corticalbasal degeneration syndrome (CBD), atypical Parkinsonian syndrome, and olivopontocerebellar degeneration (OPCD).

“Nucleobase” means an unmodified nucleobase or a modified nucleobase. As used herein “an “unmodified nucleobase” is adenine (A), thymine (T), cytosine (C), uracil (U), and guanine (G). As used herein, a “modified nucleobase” is a group of atoms other than unmodified A, T, C, U, or G capable of pairing with at least one unmodified nucleobase. A “5-methylcytosine” is a modified nucleobase. A universal base is a modified nucleobase that can pair with any one of the five unmodified nucleobases. As used herein, “nucleobase sequence” means the order of contiguous nucleobases (i.e. no additional nucleobases are present between those that are contiguous) in a nucleic acid or oligonucleotide independent of any sugar or internucleoside linkage modification.

“Nucleoside” means a compound comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. Modified nucleosides include abasic nucleosides, which lack a nucleobase. “Linked nucleosides” are nucleosides that are connected in a continuous sequence (i.e. no additional nucleosides are present between those that are linked).

“Oligomeric compound” means a compound comprising an oligonucleotide and optionally one or more additional features, such as a conjugate group or terminal group.

“Oligomeric compound” means an oligonucleotide and optionally one or more additional features, such as a conjugate group or terminal group. An oligomeric compound may be paired with a second oligomeric compound or may be unpaired. A “singled-stranded oligomeric compound” is an unpaired oligomeric compound. A “duplexed oligomeric compound” is an oligomeric compound paired with a second oligomeric compound; this is an “oligomeric duplex.”

“Oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

“Reducing or inhibiting the expression or amount” refers to a reduction or blockade of the expression or amount relative to the expression or amount in an untreated or control sample and does not necessarily indicate a total elimination of expression or amount.

“Salts” mean physiologically and pharmaceutically acceptable salts of oligomeric compounds, i.e., salts that retain the desired biological activity of the parent oligomeric compound and do not impart undesired toxicological effects thereto.

“Standard cell assay” means the assay described in Example 5 and reasonable variations thereof

“Standard in vivo experiment” means the procedure described in Example 7 and reasonable variations thereof.

“Sugar moiety” means an unmodified sugar moiety or a modified sugar moiety. As used herein, “unmodified sugar moiety” means a 2′-OH(H) furanosyl moiety, as found in RNA (an “unmodified RNA sugar moiety”), or a 2′-H(H) moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. As used herein, “modified sugar moiety” means a modified furanosyl sugar moiety or a sugar surrogate. As used herein, modified furanosyl sugar moiety means a furanosyl sugar comprising a non-hydrogen substituent in place of at least one hydrogen of an unmodified sugar moiety. Modified furanosyl sugar moieties include bicyclic sugars and non-bicyclic sugars. As used herein, “sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or nucleic acids.

“Target nucleic acid” means a nucleic acid to which an oligomeric compound is designed to hybridize.

“Therapeutically effective amount” means an amount of a pharmaceutical agent that provides a therapeutic benefit to an animal. For example, a therapeutically effective amount improves a symptom of a disease.

“Total C9ORF72 protein” means all C9ORF72 protein variants.

“Total C9ORF72 RNA” means all C9ORF72 RNA variants. “Pathogenic C9ORF72 RNA” means variants containing an expanded hexanucleotide repeat.

The present disclosure provides the following non-limiting numbered embodiments:

Embodiment 1. A method comprising administering to an animal having a neurodegenerative disease an oligomeric compound comprising a modified oligonucleotide, wherein the modified oligonucleotide consists of 12 to 30 linked nucleosides, and wherein the modified oligonucleotide has a nucleobase sequence that is at least 90% complementary to a C9ORF72 nucleic acid or a salt thereof; wherein the administering ameliorates anxiety, reduced spatial learning, or memory loss.

Embodiment 2. The method of embodiment 1, wherein the neurodegenerative disease is any of amyotrophic lateral sclerosis (ALS), frontotemporal dementia (FTD), corticalbasal degeneration syndrome (CBD), atypical Parkinsonian syndrome, and olivopontocerellar degeneration (OPCD).

Embodiment 3. The method of embodiment 1 or 2, wherein the modified oligonucleotide has a nucleobase sequence comprising at least 12, at least 13, at least 14, at least 15, at least 16, at least 17, or at least 18 contiguous nucleobases of SEQ ID NO: 6.

Embodiment 4. The method of any of embodiments 1-2, wherein the modified oligonucleotide has a nucleobase sequence of SEQ ID NO: 6.

Embodiment 5. The method of any of embodiments 1-4, wherein the modified oligonucleotide has a nucleobase sequence that is at least 90% complementary, at least 95% complementary, or 100% complementary to the nucleobase sequence of SEQ ID NO: 1 or 2, when measured across the entire nucleobase sequence of the modified oligonucleotide.

Embodiment 6. The method of any of embodiments 1-5, wherein the oligomeric compound is administered prior to the detection of the at least one symptom.

Embodiment 7. The method of any of embodiments 1-5, wherein the amelioration is the slowing of progression of at least one symptom.

Embodiment 8. The method of any of embodiments 1-5, wherein the amelioration is the delay of onset of at least one symptom.

Embodiment 9. The method of any of embodiments 1-5, wherein the amelioration is the reduction of severity of at least one symptom.

Embodiment 10. The method of any of embodiments 1-5, wherein the amelioration is the reduction of frequency of at least one symptom.

Embodiment 11. The method of any of embodiments 1-10, wherein the amount of total C9ORF72 RNA is reduced in the animal.

Embodiment 12. The method of any of embodiments 1-11, wherein the amount of total C9ORF72 protein is reduced in the animal.

Embodiment 13. The method of any of embodiments 1-10, wherein the amount of pathogenic C9ORF72 RNA is reduced in the animal.

Embodiment 14. The method of any of embodiments 1-13, wherein the animal is a human.

Embodiment 15. The method of any of embodiments 1-14, wherein the oligomeric compound is single-stranded.

Embodiment 16. The method of any of embodiment 1-15, wherein the modified oligonucleotide comprises at least one modified nucleoside.

Embodiment 17. The method of embodiment 16, wherein the modified oligonucleotide comprises at least one modified nucleoside comprising a modified sugar moiety.

Embodiment 18. The method of embodiment 17, wherein the modified oligonucleotide comprises at least one modified nucleoside comprising a bicyclic sugar moiety.

Embodiment 19. The method of embodiment 18, wherein the modified oligonucleotide comprises at least one modified nucleoside comprising a bicyclic sugar moiety having a 2′-4′ bridge, wherein the 2-4′ bridge is selected from —O—CH₂—; —O—CH₂—CH₂; and —O—CH(CH₃)—.

Embodiment 20. The method of any of embodiments 16-19, wherein the modified oligonucleotide comprises at least one modified nucleoside comprising a modified non-bicyclic sugar moiety.

Embodiment 21. The method of embodiment 20, wherein the modified oligonucleotide comprises at least one modified nucleoside comprising a non-bicyclic sugar moiety comprising a 2′-MOE or 2′-OMe.

Embodiment 22. The method of any of embodiments 17-21, wherein the modified oligonucleotide comprises at least one modified nucleoside comprising a sugar surrogate.

Embodiment 23. The method of embodiment 22, wherein the modified oligonucleotide comprises at least one modified nucleoside comprising a sugar surrogate selected from a morpholino, a PNA, a F-HNA, a THP, or a modified THP.

Embodiment 24. The method of any of embodiments 1-23, wherein the modified oligonucleotide has a sugar motif comprising:
a 5′-region consisting of 1-5 linked 5′-nucleosides;
a central region consisting of 6-10 linked central region nucleosides; and
a 3′-region consisting of 1-5 linked 3′-region nucleosides; wherein each of the 5′-region nucleosides and each of the 3′-region nucleosides comprises a modified sugar moiety and each of the central region nucleosides comprises an unmodified DNA sugar moiety.

Embodiment 25. The method of any of embodiments 1-24, wherein the modified oligonucleotide comprises at least one modified internucleoside linkage.

Embodiment 26. The method of embodiment 25, wherein each internucleoside linkage of the modified oligonucleotide is a modified internucleoside linkage.

Embodiment 27. The method of embodiment 25 or 26, wherein at least one internucleoside linkage is a phosphorothioate internucleoside linkage.

Embodiment 28. The method of embodiment 25 or 27, wherein the modified oligonucleotide comprises at least one phosphodiester internucleoside linkage.

Embodiment 29. The method of embodiment 25, wherein each internucleoside linkage is either a phosphodiester internucleoside linkage or a phosphorothioate internucleoside linkage.

Embodiment 30. The method of embodiment 26, wherein each internucleoside linkage is a phosphorothioate internucleoside linkage.

Embodiment 31. The method of any of embodiments 1-30, wherein the modified oligonucleotide comprises at least one modified nucleobase.

Embodiment 32. The method of embodiment 31, wherein the modified nucleobase is a 5-methylcytosine.

Embodiment 33. The method of any of embodiments 1-32, wherein each nucleobase of each nucleoside of the modified oligonucleotide is either an unmodified nucleobase or is a 5-methylcytosine.

Embodiment 34. The method of any of embodiments 1-33 wherein the oligomeric compound comprises a conjugate group.

Embodiment 35. The method of any of embodiments 1-14 or 16-34, wherein the oligomeric compound is paired with a second oligomeric compound to form a duplex.

I. Certain Oligonucleotides

In certain embodiments, provided herein are oligonucleotides, which consist of linked nucleosides. Oligonucleotides may be unmodified oligonucleotides (RNA or DNA) or may be modified oligonucleotides. Modified oligonucleotides comprise at least one modification relative to unmodified RNA or DNA. That is, modified oligonucleotides comprise at least one modified nucleoside (comprising a modified sugar moiety and/or a modified nucleobase) and/or at least one modified internucleoside linkage.

A. Certain Modified Nucleosides

Modified nucleosides comprise a modified sugar moiety or a modified nucleobase or both a modified sugar moiety and a modified nucleobase.

1. Certain Sugar Moieties

In certain embodiments, modified sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified sugar moieties comprising a furanosyl ring with one or more acyclic substituent, including but not limited to substituents at the 2′, 4′, and/or 5′ positions. In certain embodiments one or more acyclic substituent of non-bicyclic modified sugar moieties is branched. Examples of 2′-substituent groups suitable for non-bicyclic modified sugar moieties include but are not limited to: 2′-F, 2′-OCH₃(“OMe” or “O-methyl”), and 2′-O(CH₂)₂OCH₃ (“MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5′-methyl (R or S), 5′-vinyl, and 5′-methoxy. In certain embodiments, non-bicyclic modified sugar moieties comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836.).

In certain embodiments, a 2′-substituted nucleoside or 2′-non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted nucleoside or 2′-non-bicyclic modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted nucleoside or 2′-non-bicyclic modified nucleoside comprises a sugar moiety comprising a non-bridging 2′-substituent group selected from: F, OCH₃, and OCH₂CH₂OCH_{3.}

Nucleosides comprising modified sugar moieties, such as non-bicyclic modified sugar moieties, may be referred to by the position(s) of the substitution(s) on the sugar moiety of the nucleoside. For example, nucleosides comprising 2′-substituted sugar moieties are referred to as 2′-substituted nucleosides.

Certain modified sugar moieties comprise a bridging sugar substituent that forms a second ring resulting in a bicyclic sugar moiety. In certain such embodiments, the bicyclic sugar moiety comprises a bridge between the 4′ and the 2′ furanose ring atoms. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt” when in the S configuration), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem.,2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—;

wherein:

x is 0, 1, or 2;

n is 1, 2, 3, or 4;

each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁ acyl (C(═O)—H), substituted acyl, CN, sulfonyl (S(═O)₂-J₁), or sulfoxyl (S(═O)-J₁); and

each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 20017, 129, 8362-8379;Wengel et a., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356;Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

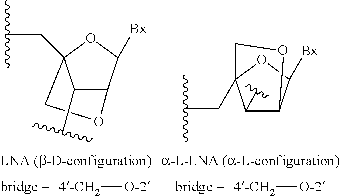

α-L-methyleneoxy (4′-CH₂—O-2′) or α-L-LNA bicyclic nucleosides have been incorporated into oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, C J. Bioorg. & Med. Chem. 2002, 10, 841-854), fluoro HNA:

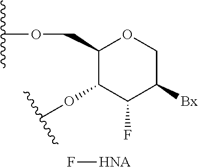

(“F-HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran), and nucleosides comprising additional modified THP compounds having the formula:

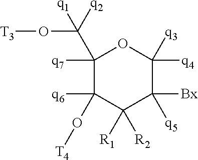

wherein, independently, for each of said modified THP nucleoside:

Bx is a nucleobase moiety;

T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3′-terminal group; q₁, q₂, q₃, q₄, q₅, q₆ and q₇ are each, independently, H, C₁-C₆ alkyl, substituted C₁C₆ alkyl, C₂-C₆ alkenyl, substituted C₂-C₆ alkenyl, C₂-C₆ alkynyl, or substituted C₂-C₆ alkynyl; and

each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

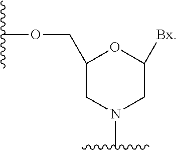

In certain embodiments, morpholinos may be modified, for example by adding or altering various substituent groups from the above morpholino structure. Such sugar surrogates are referred to herein as “modified morpholinos.”

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876.

Many other bicyclic and tricyclic sugar and sugar surrogate ring systems are known in the art that can be used in modified nucleosides).

2. Certain Modified Nucleobases

In certain embodiments, modified oligonucleotides comprise one or more nucleoside comprising an unmodified nucleobase. In certain embodiments, modified oligonucleotides comprise one or more nucleoside comprising a modified nucleobase. In certain embodiments, modified oligonucleotides comprise one or more nucleoside that does not comprise a nucleobase, referred to as an abasic nucleoside.

In certain embodiments, modified nucleobases are selected from: 5-substituted pyrimidines, 6-azapyrimidines, alkyl or alkynyl substituted pyrimidines, alkyl substituted purines, and N-2, N-6 and O-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine , 2-thiouracil, 2-thiothymine and 2-thiocytosine, 5-propynyl (—C═C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manohara et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. NO. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., U.S. Pat. No. 6,166,199; and Matteucci et al., U.S. Pat. No. 6,005,096.

3. Certain Modified Internucleoside Linkages

In certain embodiments, nucleosides of modified oligonucleotides may be linked together using any internucleoside linkage. The two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS—P═S”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. In certain embodiments, internucleoside linkages having a chiral atom can be prepared as a racemic mixture, or as separate enantiomers. Representative chiral internucleoside linkages include but are not limited to alkylphosphonates and phosphorothioates. Methods of preparation of phosphorous-containing and non-phosphorous-containing internucleoside linkages are well known to those skilled in the art.

Neutral internucleoside linkages include, without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl, and thioformacetal (3′-S—CH₂-O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

B. Certain Motifs

In certain embodiments, modified oligonucleotides comprise one or more modified nucleosides comprising a modified sugar moiety. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases). 1. Certain Sugar Motifs

In certain embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

In certain embodiments, modified oligonucleotides comprise or consist of a region having a gapmer motif, which comprises two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-5 nucleosides. In certain embodiments, each nucleoside of each wing of a gapmer is a modified nucleoside.

In certain embodiments, the gap of a gapmer comprises 7-12 nucleosides. In certain embodiments, each nucleoside of the gap of a gapmer is an unmodified 2′-deoxy nucleoside.

In certain embodiments, the gapmer is a deoxy gapmer. In embodiments, the nucleosides on the gap side of each wing/gap junction are unmodified 2′-deoxy nucleosides and the nucleosides on the wing sides of each wing/gap junction are modified nucleosides. In certain embodiments, each nucleoside of the gap is an unmodified 2′-deoxy nucleoside. In certain embodiments, each nucleoside of each wing of a gapmer is a modified nucleoside.

In certain embodiments, modified oligonucleotides comprise or consist of a region having a fully modified sugar motif. In such embodiments, each nucleoside of the fully modified region of the modified oligonucleotide comprises a modified sugar moiety. In certain embodiments, each nucleoside of the entire modified oligonucleotide comprises a modified sugar moiety. In certain embodiments, modified oligonucleotides comprise or consist of a region having a fully modified sugar motif, wherein each nucleoside within the fully modified region comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified comprises the same 2′-modification.

2. Certain Nucleobase Motifs

In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified.

In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of said nucleoside is a 2′-deoxyribosyl moiety. In certain embodiments, the modified nucleobase is selected from: a 2-thiopyrimidine and a 5-propynepyrimidine.

3. Certain Internucleoside Linkage Motifs

In certain embodiments, oligonucleotides comprise modified and/or unmodified internucleoside linkages arranged along the oligonucleotide or region thereof in a defined pattern or motif. In certain embodiments, each internucleoside linking group is a phosphodiester internucleoside linkage (P═O). In certain embodiments, each internucleoside linking group of a modified oligonucleotide is a phosphorothioate internucleoside linkage (P═S). In certain embodiments, each internucleoside linkage of a modified oligonucleotide is independently selected from a phosphorothioate internucleoside linkage and phosphodiester internucleoside linkage. In certain embodiments, the sugar motif of a modified oligonucleotide is a gapmer and the internucleoside linkages within the gap are all modified. In certain such embodiments, some or all of the internucleoside linkages in the wings are unmodified phosphate linkages. In certain embodiments, the terminal internucleoside linkages are modified.

C. Certain Lengths

It is possible to increase or decrease the length of an oligonuclotide without eliminating activity. For example, in Woolf et al. (Proc. Natl. Acad. Sci. USA 89:7305-7309, 1992), a series of oligonucleotides 13-25 nucleobases in length were tested for their ability to induce cleavage of a target RNA in an oocyte injection model. Oligonucleotides 25 nucleobases in length with 8 or 11 mismatch bases near the ends of the oligonucleotides were able to direct specific cleavage of the target mRNA, albeit to a lesser extent than the oligonucleotides that contained no mismatches. Similarly, target specific cleavage was achieved using 13 nucleobase oligonucleotides, including those with 1 or 3 mismatches.

In certain embodiments, oligonucleotides (including modified oligonucleotides) can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29,20 to 30,21 to 22, 21 to 23, 21 to 24, 21 to 25,21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides

D. Certain Modified Oligonucleotides

In certain embodiments, the above modifications (sugar, nucleobase, internucleoside linkage) are incorporated into a modified oligonucleotide. In certain embodiments, modified oligonucleotides are characterized by their modification motifs and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such sugar gapmer oligonucleotides may comprise one or more modified nucleobase independent of the gapmer pattern of the sugar modifications. Unless otherwise indicated, all modifications are independent of nucleobase sequence.

E. Nucleobase Sequence

In certain embodiments, oligonucleotides (unmodified or modified oligonucleotides) are further described by their nucleobase sequence. In certain embodiments oligonucleotides have a nucleobase sequence that is complementary to a second oligonucleotide or an identified reference nucleic acid, such as a target nucleic acid. In certain such embodiments, a region of an oligonucleotide has a nucleobase sequence that is complementary to a second oligonucleotide or an identified reference nucleic acid, such as a target nucleic acid. In certain embodiments, the nucleobase sequence of a region or entire length of an oligonucleotide is at least 50%, at least 60%, at least 70%, at least 80%, at least 85%, at least 90%, at least 95%, or 100% complementary to the second oligonucleotide or nucleic acid, such as a target nucleic acid.

II. Certain Oligomeric Compounds

In certain embodiments, the invention provides oligomeric compounds, which consist of an oligonucleotide (modified or unmodified) and optionally one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, modified or unmodified nucleosides, and two or more nucleosides that are independently modified or unmodified.

A. Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance. In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

1. Conjugate Moieties

Conjugate moieties include, without limitation, intercalators, reporter molecules, polyamines, polyamides, peptides, carbohydrates, vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

2. Conjugate Linkers

Conjugate moieties are attached to oligonucleotides through conjugate linkers. In certain oligomeric compounds, the conjugate linker is a single chemical bond (i.e., the conjugate moiety is attached directly to an oligonucleotide through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a parent compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise exactly 3 linker-nucleosides. In certain embodiments, conjugate linkers comprise the TCA motif In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodimements, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an oligomeric compound is more than 30. Alternatively, an oligomeric compound may comprise a modified oligonucleotide consisting of 8-30 nucleosides and no conjugate group. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases. In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxy nucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

III. Duplexed Oligomeric Compounds

In certain embodiments, oligomeric compounds described herein comprise an oligonucleotide, having a nucleobase sequence complementary to that of a target nucleic acid. In certain embodiments, an oligomeric compound is paired with a second oligomeric compound to form an oligomeric duplex. Such oligomeric duplexes comprise a first oligomeric compound having a region complementary to a target nucleic acid and a second oligomeric compound having a region complementary to the first oligomeric compound. In certain embodiments, the first oligomeric compound of an oligomeric duplex comprises or consists of (1) a modified or unmodified oligonucleotide and optionally a conjugate group and (2) a second modified or unmodified oligonucleotide and optionally a conjugate group. Either or both oligomeric compounds of an oligomeric duplex may comprise a conjugate group. The oligonucleotides of each oligomeric compound of an oligomeric duplex may include non-complementary overhanging nucleosides.

IV. Antisense Activity

In certain embodiments, oligomeric compounds and oligomeric duplexes are capable of hybridizing to a target nucleic acid, resulting in at least one antisense activity; such oligomeric compounds and oligomeric duplexes are antisense compounds. In certain embodiments, antisense compounds selectively affect one or more target nucleic acid. Such antisense compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in significant undesired antisense activity.

In certain antisense activities, hybridization of an antisense compound to a target nucleic acid results in recruitment of a protein that cleaves the target nucleic acid. For example, certain antisense compounds result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, described herein are antisense compounds that are sufficiently “DNA-like” to elicit RNase H activity. In certain embodiments, one or more non-DNA-like nucleoside in the gap of a gapmer is tolerated.

In certain antisense activities, an antisense compound or a portion of an antisense compound is loaded into an RNA-induced silencing complex (RISC), ultimately resulting in cleavage of the target nucleic acid. For example, certain antisense compounds result in cleavage of the target nucleic acid by Argonaute. Antisense compounds that are loaded into RISC are RNAi compounds. RNAi compounds may be double-stranded (siRNA) or single-stranded (ssRNA).

In certain embodiments, hybridization of an antisense compound to a target nucleic acid does not result in recruitment of a protein that cleaves that target nucleic acid. In certain embodiments, hybridization of the antisense compound to the target nucleic acid results in alteration of splicing of the target nucleic acid. In certain embodiments, hybridization of an antisense compound to a target nucleic acid results in inhibition of a binding interaction between the target nucleic acid and a protein or other nucleic acid. In certain embodiments, hybridization of an antisense compound to a target nucleic acid results in alteration or translation of the target nucleic acid.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein, and/or a phenotypic change in a cell or animal.

V. Certain Target Nucleic Acids

In certain embodiments, oligomeric compounds comprise or consist of an oligonucleotide comprising a region that is complementary to a target nucleic acid. In certain embodiments, the target nucleic acid is an endogenous RNA molecule. In certain embodiments, the target nucleic acid encodes a protein. In certain such embodiments, the target nucleic acid is selected from: a mature mRNA and a pre-mRNA, including intronic, exonic and untranslated regions. In certain embodiments, the target RNA is a mature mRNA. In certain embodiments, the target nucleic acid is a pre-mRNA. In certain such embodiments, the target region is entirely within an intron. In certain embodiments, the target region spans an intron/exon junction. In certain embodiments, the target region is at least 50% within an intron.

In certain embodiments, the target nucleic acid is a non-coding RNA. In certain such embodiments, the target non-coding RNA is selected from: a long-non-coding RNA, a short non-coding RNA, an intronic RNA molecule, a snoRNA, a scaRNA, a microRNA (including pre-microRNA and mature microRNA), a ribosomal RNA, and promoter directed RNA. In certain embodiments, the target nucleic acid is a nucleic acid other than a mature mRNA. In certain embodiments, the target nucleic acid is a nucleic acid other than a mature mRNA or a microRNA. In certain embodiments, the target nucleic acid is a non-coding RNA other than a microRNA. In certain embodiments, the target nucleic acid is a non-coding RNA other than a microRNA or an intronic region of a pre-mRNA. In certain embodiments, the target nucleic acid is a long non-coding RNA. In certain embodiments, the target nucleic acid is a non-coding RNA associated with splicing of other pre-mRNAs. In certain embodiments, the target nucleic acid is a nuclear-retained non-coding RNA.

In certain embodiments, oligomeric compounds described herein are complementary to a target nucleic acid comprising a single-nucleotide polymorphism (SNP). In certain such embodiments, the oligomeric compound is capable of modulating expression of one allele of the SNP-containing target nucleic acid to a greater or lesser extent than it modulates another allele. In certain embodiments, an oligomeric compound hybridizes to a (SNP)-containing target nucleic acid at the single-nucleotide polymorphism site.

In certain embodiments, oligomeric compounds are at least partially complementary to more than one target nucleic acid. For example, oliogomeric compounds described herein may mimic microRNAs, which typically bind to multiple targets.

A. Complementarity/Mismatches to the Target Nucleic Acid

It is possible to introduce mismatch bases without eliminating activity. For example, Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo. Maher and Dolnick (Nuc. Acid. Res. 16:3341-3358, 1988) tested a series of tandem 14 nucleobase oligonucleotides, and a 28 and 42 nucleobase oligonucleotides comprised of the sequence of two or three of the tandem oligonucleotides, respectively, for their ability to arrest translation of human DHFR in a rabbit reticulocyte assay. Each of the three 14 nucleobase oligonucleotides alone was able to inhibit translation, albeit at a more modest level than the 28 or 42 nucleobase oligonucleotides.

In certain embodiments, oligomeric compounds comprise oligonucleotides that are complementary to the target nucleic acid over the entire length of the oligonucleotide. In certain embodiments, oligonucleotides are 99%, 95%, 90%, 85%, or 80% complementary to the target nucleic acid. In certain embodiments, oligonucleotides are at least 80% complementary to the target nucleic acid over the entire length of the oligonucleotide and comprise a region that is 100% or fully complementary to a target nucleic acid. In certain embodiments, the region of full complementarity is from 6 to 20, 10 to 18, or 18 to 20 nucleobases in length.

In certain embodiments, oligonucleotides comprise one or more mismatched nucleobases relative to the target nucleic acid. In certain embodiments, antisense activity against the target is reduced by such mismatch, but activity against a non-target is reduced by a greater amount. Thus, in certain embodiments selectivity of the oligomeric compound comprising an oligonucleotide is improved. In certain embodiments, the mismatch is specifically positioned within an oligonucleotide having a gapmer motif. In certain embodiments, the mismatch is at position 1, 2, 3, 4, 5, 6, 7, or 8 from the 5′-end of the gap region. In certain embodiments, the mismatch is at position 9, 8, 7, 6, 5, 4, 3, 2, 1 from the 3′-end of the gap region. In certain embodiments, the mismatch is at position 1, 2, 3, or 4 from the 5′-end of the wing region. In certain embodiments, the mismatch is at position 4, 3, 2, or 1 from the 3′-end of the wing region.

B. C9ORF72

In certain embodiments, oligomeric compounds comprise or consist of any oligonucleotide comprising a region that is complementary to a target nucleic acid, wherein the target nucleic acid is C9ORF72. In certain embodiments, C9ORF72 nucleic acid has the sequence set forth in GENBANK Accession No. NM_018325.4 (incorporated herein as SEQ ID NO: 1) or the complement of GENBANK Accession No. NT_008413.18 truncated from nucleobase 27535000 to 27565000 (incorporated herein as SEQ ID NO: 2).

In certain embodiments, contacting a cell with an oligonucleotide complementary to SEQ ID NO: 1 or SEQ ID NO: 2 reduces the amount of C9ORF72 RNA. In certain embodiments, contacting a cell with an oligonucleotide complementary to SEQ ID NO: 1 or SEQ ID NO: 2 reduces the amount of total C9ORF72 protein. In certain embodiments, contacting a cell with an oligomeric compound complementary to SEQ ID NO: 1 or SEQ ID NO: 2 ameliroates one or more symptoms of a neurodegenerative disease. In certain embodiments, the symptom is anxiety, reduced spatial learning, and memory loss.

C. Certain Target Nucleic Acids in Certain Tissues

In certain embodiments, oligomeric compounds comprise or consist of an oligonucleotide comprising a region that is complementary to a target nucleic acid, wherein the target nucleic acid is expressed in CNS tissue, including spinal cord and cortex.

VI. Certain Pharmaceutical Compositions

In certain embodiments, described herein are pharmaceutical compositions comprising one or more oligomeric compounds or a salt thereof. In certain embodiments, the pharmaceutical composition comprises a pharmaceutically acceptable diluent or carrier. In certain embodiments, a pharmaceutical composition comprises a sterile saline solution and one or more oligomeric compound. In certain embodiments, a pharmaceutical composition consists of a sterile saline solution and one or more oligomeric compound. In certain embodiments, the sterile saline is pharmaceutical grade saline. In certain embodiments, a pharmaceutical composition comprises one or more oligomeric compound and sterile water. In certain embodiments, a pharmaceutical composition consists of one oligomeric compound and sterile water. In certain embodiments, the sterile water is pharmaceutical grade water. In certain embodiments, a pharmaceutical composition comprises one or more oligomeric compound and phosphate-buffered saline (PBS). In certain embodiments, a pharmaceutical composition consists of one or more oligomeric compound and sterile PBS. In certain embodiments, the sterile PBS is pharmaceutical grade PBS.

In certain embodiments, pharmaceutical compositions comprise one or more oligomeric compound and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, oligomeric compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising an oligomeric compound encompass any pharmaceutically acceptable salts of the oligomeric compound, esters of the oligomeric compound, or salts of such esters. In certain embodiments, pharmaceutical compositions comprising oligomeric compounds comprising one or more oligonucleotide, upon administration to an animal, including a human, are capable of providing (directly or indirectly) the biologically active metabolite or residue thereof. Accordingly, for example, the disclosure is also drawn to pharmaceutically acceptable salts of oligomeric compounds, prodrugs, pharmaceutically acceptable salts of such prodrugs, and other bioequivalents. Suitable pharmaceutically acceptable salts include, but are not limited to, sodium and potassium salts. In certain embodiments, prodrugs comprise one or more conjugate group attached to an oligonucleotide, wherein the conjugate group is cleaved by endogenous nucleases within the body.

Lipid moieties have been used in nucleic acid therapies in a variety of methods. In certain such methods, the nucleic acid, such as an oligomeric compound, is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver the one or more pharmaceutical agents of the present invention to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal, intracerebroventricular, etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain.

### Nonlimiting Disclosure and Incorporation by Reference

Each of the literature and patent publications listed herein is incorporated by reference in its entirety.

While certain compounds, compositions and methods described herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH in place of one 2′-H of DNA) or as an RNA having a modified base (thymine (methylated uracil) in place of a uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “AT^{m}CGAUCG,” wherein mC indicates a cytosine base comprising a methyl group at the 5-position.

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as a or 13 such as for sugar anomers, or as (D) or (L), such as for amino acids, etc. Included in the compounds provided herein are all such possible isomers, including their racemic and optically pure forms, unless specified otherwise. Likewise, all cis- and trans-isomers and tautomeric forms are also included unless otherwise indicated. Unless otherwise indicated, compounds described herein are intended to include corresponding salt forms.

### EXAMPLES

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif. And, for example, where a particular high-affinity modification appears at a particular position, other high-affinity modifications at the same position are considered suitable, unless otherwise indicated.

### Example 1

### Modified Oligonucleotides Targeting Human C9ORF72

The modified oligonucleotide in the table below is a MOE gapmer. The central gap segment of the gapmer contains 2′-deoxynucleosides and is flanked by wing segments on both the 5′ end and on the 3′ end containing nucleosides that each comprise a 2′-MOE group. The specific motif of the gapmer is listed in table below, represented by three numbers separated by hyphens. The numbers represent the number of nucleosides in the 5′-wing, the gap, and the 3′-wing, respectively. All cytosine residues throughout the oligonucleotide are 5-methylcytosines. The internucleoside linkages for the gapmer are mixed phosphorothioate and phosphodiester linkages. The internucleoside linkages for the gapmer are presented in the Linkage column, where ‘o’ indicates a phosphodiester linkage and ‘s’ indicates a phosphorothioate linkage.

The modified oligonucleotide listed in the table below is targeted to the human C9ORF72 genomic sequence, designated herein as SEQ ID NO: 2 (the complement of GENBANK Accession No. NT_008413.18 truncated from nucleosides 27535000 to 27565000). “Start site” indicates the 5′-most nucleoside to which the gapmer is targeted in the human genomic sequence. “Stop site” indicates the 3′-most nucleoside to which the gapmer is targeted human genomic sequence.

TABLE 1
Modified oligonucleotides targeting
human C9ORF72

| | | | Start | Stop | | |
|---|---|---|---|---|---|---|
| | | | Site | Site | | |
| | | | SEQ | SEQ | | |
| Com- | | | ID | ID | | SEQ |
| pound | | | NO: | NO: | | ID |
| No | Sequence | Linkage | 2 | 2 | Motif | NO |
| 672681 | GCCCCTAGC | sooosssss | 1444 | 1461 | 5-8-5 | 6 |
| | GCGCGACTC | ssssooss | | | | |

### Example 2

### In Vitro Dose Response Inhibition of Human Pathogenic C9ORF72 RNA in HepG2 Cells with 672681

Compound No. 672681 was tested at various doses in HepG2 cells. Cells were plated at a density of 20,000 cells per well and transfected using electroporation with 0.11 μM, 0.33 μM, 1.00 μM, or 3.00 μM concentrations of modified oligonucleotide. After a treatment period of approximately 16 hours, RNA was isolated from the cells and C9ORF72 mRNA levels were measured by quantitative real-time PCR using human primer probe set RTS3905 (forward primer: GGGTCTAGCAAGAGCAGGTG, designated herein as SEQ ID NO: 3; reverse primer: GTCTTGGCAACAGCTGGAGAT, designated herein as SEQ ID NO: 4; probe: TGATGTCGACTCTTTGCCCACCGC, designated herein as SEQ ID NO: 5-a TAQ-man primer probe set). Primer probe set RTS3905 was used to measure the pathogenic C9ORF72 RNA, which is the product of a pre-mRNA containing a hexanucleotide repeat. C9ORF72 mRNA levels were adjusted according to total RNA content, as measured by RIBOGREEN®. Results are presented in Table 2 below as percent inhibition of human C9ORF72 mRNA levels, relative to untreated control cells.

TABLE 2
Percent inhibition of the pathogenic C9ORF72 RNA in HepG2 cells

| Compound No | 0.11 μM | 0.33 μM | 1.00 μM | 3.00 μM |
|---|---|---|---|---|
| 672681 | 27 | 42 | 63 | 87 |

### Example 3

### Inhibition of Human C9ORF72 RNA in Transgenic Mice

Inhibition of C9ORF72 RNA was tested using Compound No. 672681 in a BAC transgenic mouse line, designated herein as C9B183. The C9B183 mouse line expresses a truncated human C9ORF72 gene comprising exons 1-5. The truncated human C9ORF72 gene of the C9B183 mouse line contains 450 hexanucleotide repeats.

3-month old C9B183 mice each received a single intra cerebroventricular (ICV) stereotactic injection into the right ventricle of 350 μg of either Compound No. 672681 or a control oligonucleotide that does not target human C9ORF72 in 10 μL total volume of PBS. Each treatment group consisted of six mice. Two weeks following oligonucleotide treatment, the mice were sacrificed and spinal cord and cortex tissues were collected from each mouse. Total RNA from each tissue was isolated with TRIZOL (Invitrogen, Carlsbad, Calif.) and first-strand cDNA was synthesized using the SuperScript III First-strand synthesis kit (Invitrogen, Carlsbad, Calif.). RT-qPCR of human pathogenic C9ORF72 RNA was performed using primer probe set RTS3905 (see Example 2) and normalized to GAPDH. The average results for each treatment group are presented in the table below as normalized percent inhibition of human C9ORF72 RNA relative to the control treated mice.

TABLE 3
Percent inhibition of human pathogenic C9ORF72 RNA in vivo

Human C9ORF72 RNA (%)

| Oligonucleotide | Cortex | Spinal Cord |
|---|---|---|
| Control | 100 | 100 |
| Isis No. 672681 | 23 | 12 |

### Example 4

### Behavioral Effects of Modified Oligonucleotide-Mediated Inhibition of Human C9ORF72 RNA

Nine month old wild type and C9B183 mice each received a single ICV stereotactic injection into the right ventricle of 350 μg of either Isis No. 672681 or a control oligonucleotide that does not target human C9ORF72 in 10 μL total volume of PBS. Each treatment group consisted of ten to thirteen mice. At 12 months of age and 15 months of age, the anxiety-related and cognition-related behaviors described below were assessed, except for the radial arm maze, which was assessed only at 12 months.

### Marble Burying

Mice were placed individually in a standard mouse cage containing bedding that is 5 cm in depth, with 20 small marbles arranged in 4 evenly spaced rows of 5 on top of the bedding material. After 20 minutes, mice were removed and the number of marbles buried was determined (marbles that were at least ⅔ covered by bedding were counted as buried). Increased marble burying is associated with increased anxiety-related behavior. The results are presented in the table below as the average percentage of marbles buried for each treatment group.

### Elevated Plus Maze

The plus maze apparatus had four arms (5×30 cm) at right angles to each other and was elevated 30 cm from the floor. Two of the arms had 16 cm high walls (enclosed arms) and two arms had a 0.5 cm lip, but no walls (open arms). Mice were placed onto the center of the maze and allowed free access to all four arms for 5 minutes. Behavior was recorded using a camera mounted above the apparatus. An increased percentage of time spent on the enclosed arms is associated with increased anxiety-related behavior. The results are presented in the table below as the average percentage of time spent in the open arms for each treatment group.

### Barnes Maze

An opaque Plexiglas disc 75 cm in diameter and elevated 58 cm above the floor on a tripod was used as the Barnes Maze. Twenty holes, 5 cm in diameter each, were located 5 cm from the perimeter, and a black Plexiglas escape box (19×8×7 cm) was placed under one of the holes. Distinct spatial cues were located around the maze and were kept constant throughout the study. On the first day of testing, a training session was performed, in which the mouse was placed in the escape box for five minutes. One minute later, the first session was started. At the beginning of each session, the mouse was placed in the middle of the platform in a 10 cm high cylindrical black start chamber. After 10 seconds, the start chamber was removed, a light (400 lux) was turned on, and the mouse was allowed to freely explore the maze. The session ended when the mouse entered the escape tunnel or after 3 minutes had elapsed. When the mouse entered the escape tunnel, the light was turned off and the mouse remained in the dark for one minute. When the mouse did not enter the tunnel by itself it was gently put in the escape box for one minute. The tunnel was always located underneath the same hole (stable within the spatial environment), which was randomly determined for each mouse. Mice were tested once a day for 9 days. The number of errors in finding the escape chamber one each day of testing was tabulated for each mouse. The results are presented in the table below as the average number of errors for each treatment group over days 7 through 9 of testing. The Barnes maze is a measure of spatial learning and memory.

### Radial Arm Maze

Prior to the start of the study, all animals underwent a 5 day period of food restriction wherein their body weights were reduced to 90% of the original weight by restricting available food to 2 grams of chow per mouse. Once each animal reached the target weight of 90% of original body weight, food rations were adjusted accordingly to this weight. The purpose of the food deprivation was to establish food as a motivator. Next, mice were trained daily on an elevated, 8 arm-radial arm maze, with a circular recessed food cup located at the end of each arm. The first 3 days served as a familiarization period for the mice. On the first day, each mouse was placed on the maze for 10 minutes with no food present. On the next two days, food was scattered on the center platform and over the entire length of four of the arms of the maze. The four arms containing food were distributed randomly throughout the maze (e.g., arms 1, 4, 5, and 7 were baited with food). After completing the familiarization phase, food pellets were placed only in the food cups at the end of four designated arms. Each mouse remained on the maze until it found the four food cups or until 10 minutes had elapsed. During each trial, the number and order of arms entered by each mouse was recorded. Entries into arms that had never contained food following the familiarization phase were considered errors in reference memory. Re-entering a previously entered arm was considered an error in working memory. The results are presented in the table below as the average total number of errors for each treatment group on the indicated day of testing.

TABLE 4
Behavioral effects of modified oligonucleotide-
mediated inhibition of human C9ORF72 RNA in mice

| | Marble | | Elevated Plus | | | | | | |
|---|---|---|---|---|---|---|---|---|---|
| | Burying | | Maze (% time | | Barnes Maze | | Radial Arm Maze | | |
| | (% buried) | | on open arms) | | (No. of errors) | | (No. errors) | | |
| | 12 | 15 | 12 | 15 | 12 | 15 | Day | Day | Day |
| Group | months | months | months | months | months | months | 8 | 9 | 10 |
| WT; | 51 | 33 | 26.4 | 19.0 | 13.0 | 14.3 | 4.1 | 3.7 | 2.3 |
| control | | | | | | | | | |
| WT; | 50 | 38 | 24.5 | 17.3 | 12.2 | 15.9 | 4.0 | 3.8 | 2.6 |
| Isis No. | | | | | | | | | |
| 672681 | | | | | | | | | |
| C9B183; | 68 | 63 | 14.4 | 7.7 | 20.7 | 23.5 | 6.1 | 6.3 | 6.7 |
| control | | | | | | | | | |
| C9B183; | 48 | 39 | 23.6 | 19.9 | 16.7 | 18.4 | 4.9 | 4.9 | 4.5 |
| Isis No. | | | | | | | | | |
| 672681 | | | | | | | | | |

1. A method comprising administering to an animal having a neurodegenerative disease an oligomeric compound comprising a modified oligonucleotide, wherein the modified oligonucleotide consists of 12 to 30 linked nucleosides, and wherein the modified oligonucleotide has a nucleobase sequence that is at least 90% complementary to a C9ORF72 nucleic acid or a salt thereof; wherein the administering ameliorates anxiety, reduced spatial learning, or memory loss.

2. The method of claim 1, wherein the neurodegenerative disease is any of amyotrophic lateral sclerosis (ALS), frontotemporal dementia (FTD), corticalbasal degeneration syndrome (CBD), atypical Parkinsonian syndrome, and olivopontocerellar degeneration (OPCD).

3. The method of claim 1 or 2, wherein the modified oligonucleotide has a nucleobase sequence comprising at least 12, at least 13, at least 14, at least 15, at least 16, at least 17, or at least 18 contiguous nucleobases of SEQ ID NO: 6.

4. The method of any of claims 1-2, wherein the modified oligonucleotide has a nucleobase sequence of SEQ ID NO: 6.

5. The method of any of claims 1-4, wherein the modified oligonucleotide has a nucleobase sequence that is at least 90% complementary, at least 95% complementary, or 100% complementary to the nucleobase sequence of SEQ ID NO: 1 or 2, when measured across the entire nucleobase sequence of the modified oligonucleotide.

6. The method of any of claims 1-5, wherein the oligomeric compound is administered prior to the detection of the at least one symptom.

7. The method of any of claims 1-5, wherein the amelioration is the slowing of progression of at least one symptom.

8. The method of any of claims 1-5, wherein the amelioration is the delay of onset of at least one symptom.

9. The method of any of claims 1-5, wherein the amelioration is the reduction of severity of at least one symptom.

10. The method of any of claims 1-5, wherein the amelioration is the reduction of frequency of at least one symptom.

11. The method of any of claims 1-10, wherein the amount of total C9ORF72 RNA is reduced in the animal.

12. The method of any of claims 1-11, wherein the amount of total C9ORF72 protein is reduced in the animal.

13. The method of any of claims 1-10, wherein the amount of pathogenic C9ORF72 RNA is reduced in the animal.

14. The method of any of claims 1-13, wherein the animal is a human.

15. The method of any of claims 1-14, wherein the oligomeric compound is single-stranded.

16. The method of any of claim 1-15, wherein the modified oligonucleotide comprises at least one modified nucleoside.

17. The method of claim 16, wherein the modified oligonucleotide comprises at least one modified nucleoside comprising a modified sugar moiety.

18. The method of claim 17, wherein the modified oligonucleotide comprises at least one modified nucleoside comprising a bicyclic sugar moiety.

19. The method of claim 18, wherein the modified oligonucleotide comprises at least one modified nucleoside comprising a bicyclic sugar moiety having a 2′-4′ bridge, wherein the 2-4′ bridge is selected from —O—CH₂—; —O—CH₂—CH₂; and —O—CH(CH₃)—.

20. The method of any of claims 16-19, wherein the modified oligonucleotide comprises at least one modified nucleoside comprising a modified non-bicyclic sugar moiety.

21. The method of claim 20, wherein the modified oligonucleotide comprises at least one modified nucleoside comprising a non-bicyclic sugar moiety comprising a 2′-MOE or 2′-OMe.

22. The method of any of claims 17-21, wherein the modified oligonucleotide comprises at least one modified nucleoside comprising a sugar surrogate.

23. The method of claim 22, wherein the modified oligonucleotide comprises at least one modified nucleoside comprising a sugar surrogate selected from a morpholino, a PNA, a F-HNA, a THP, or a modified THP.

24. The method of any of claims 1-23, wherein the modified oligonucleotide has a sugar motif comprising:
a 5′-region consisting of 1-5 linked 5′-nucleosides;
a central region consisting of 6-10 linked central region nucleosides; and
a 3′-region consisting of 1-5 linked 3′-region nucleosides; wherein each of the 5′-region nucleosides and each of the 3′-region nucleosides comprises a modified sugar moiety and each of the central region nucleosides comprises an unmodified DNA sugar moiety.

25. The method of any of claims 1-24, wherein the modified oligonucleotide comprises at least one modified internucleoside linkage.

26. The method of claim 25, wherein each internucleoside linkage of the modified oligonucleotide is a modified internucleoside linkage.

27. The method of claim 25 or 26, wherein at least one internucleoside linkage is a phosphorothioate internucleoside linkage.

28. The method of claim 25 or 27, wherein the modified oligonucleotide comprises at least one phosphodiester internucleoside linkage.

29. The method of claim 25, wherein each internucleoside linkage is either a phosphodiester internucleoside linkage or a phosphorothioate internucleoside linkage.

30. The method of claim 26, wherein each internucleoside linkage is a phosphorothioate internucleoside linkage.

31. The method of any of claims 1-30, wherein the modified oligonucleotide comprises at least one modified nucleobase.

32. The method of claim 31, wherein the modified nucleobase is a 5-methylcytosine.

33. The method of any of claims 1-32, wherein each nucleobase of each nucleoside of the modified oligonucleotide is either an unmodified nucleobase or is a 5-methylcytosine.

34. The method of any of claims 1-33 wherein the oligomeric compound comprises a conjugate group.

35. The method of any of claim 1-14 or 16-34, wherein the oligomeric compound is paired with a second oligomeric compound to form a duplex.
